(19) United States
(12) Patent Application Publication
(10) Pub. No.: US 2023/0165498 A1
(43) Pub. Date: Jun. 1, 2023

(54) Alertness Services

(71) Applicant: ResMed Sensor Technologies Limited, Dublin (IE)

(72) Inventor: Redmond Shouldice, Dublin (IE)

(21) Appl. No.: 17/997,488

(22) PCT Filed: Apr. 28, 2021
(86) PCT No.: PCT/IB2021/053550

§ 371 Date: Oct. 28, 2022

Related U.S. Application Data
(60) Provisional application No. 63/018,323, filed on Apr. 30, 2020.

(51) Int. Cl. A61B 5/16; A61B 5/00; A61B 5/11

(52) U.S. Cl. CPC A61B 5/168; A61B 5/7267; A61B 5/746; A61B 5/11; A61B 5/4806; A61M 2205/50

(57) ABSTRACT

User alertness can be monitored and leveraged while the user is interacting with a computing device, such as a mobile device (e.g., a smartphone or tablet). By monitoring interaction data, an alertness inference of the user can be generated. The interaction data can include biometric data of the user (e.g., blink rate, eye focus, and breathing rate), inertia data of the device (e.g., swaying and orientation), and software-usage data of the device (e.g., button press speed and accuracy, app or action being used, and response times). The alertness inference can be a score measuring a degree of alertness of the user, from a deep sleep through fully alert. The alertness inference can be leveraged to automatically alert presentation of a message (e.g., notification) on the device, such as withholding presentation of the message or presenting it in different fashion (e.g., silently).

### CROSS-REFERENCE TO RELATED APPLICATIONS

This application claims the benefit of, and priority to, U.S. Provisional Patent Application No. 63/018,323, filed Apr. 30, 2020, which is hereby incorporated by reference herein in its entirety.

### TECHNICAL FIELD

The present disclosure relates to computing devices generally and more specifically to controlling computing devices using alertness inferences.

### BACKGROUND

Many computing devices, such as smartphones, tablets, and laptops, are used at various times of day, in various conditions, and for various purposes. For example, a user may interact with such devices early in the morning when waking, throughout the day for work or non-work purposes, during the evening, and at night, such as prior to falling asleep. Throughout the day, or throughout a session during which the user is using a computing device, the user may exhibit different degrees of alertness when interacting with these devices. For example, the user may be alert and attentive during the day, but may be losing alertness at night prior to falling asleep. In a further example, the user may be alert and attentive during an early period of a session during which the user is using the computing device, but may be losing alertness after this period. In another example, a user on a bus or train may lose alertness during the journey, potentially falling asleep or otherwise lightly dozing off prior to their destination.

Depending on the level of alertness, the user's ability to interact with the computing device may vary, as well as their ability and desire to interact with certain features of the device. Additionally, certain features of the device may harm the user's ability to concentrate or sleep in various situations when concentration or sleep are needed.

Current technologies generally rely on preset rules, such as do-not-disturb settings, which may be set to turn on at certain times of day and may turn off at a certain time of day or when the user presses a button to dismiss those settings. However, while these types of rules attempt to engage at appropriate times, they are not related to the user's actual alertness state. Therefore, a notification may be withheld if the do-not-disturb setting happens to be on, regardless of the user's actual alertness and ability or desire to receive the notification. Likewise, if a user is not alert and may not desire to receive a notification, the notification may be presented nonetheless if the do-not-disturb setting happens to not be on.

Thus, there is a need to improve functionality of computing devices to be able to adapt to a user's alertness level. In particular, there is a need to minimize unwanted or sleep-damaging messages and notifications. There is also a need to facilitate delivery of messages and notification at appropriate times.

### SUMMARY

The term embodiment and like terms are intended to refer broadly to all of the subject matter of this disclosure and the claims below. Statements containing these terms should be understood not to limit the subject matter described herein or to limit the meaning or scope of the claims below. Embodiments of the present disclosure covered herein are defined by the claims below, supplemented by this summary. This summary is a high-level overview of various aspects of the disclosure and introduces some of the concepts that are further described in the Detailed Description section below. This summary is not intended to identify key or essential features of the claimed subject matter, nor is it intended to be used in isolation to determine the scope of the claimed subject matter. The subject matter should be understood by reference to appropriate portions of the entire specification of this disclosure, any or all drawings and each claim.

Embodiments of the present disclosure include a method comprising receiving a first message intended for presentation by a computing device; presenting the first message on the computing device using a first presentation scheme in response to receiving the first message; receiving interaction data associated with a user interacting with the computing device; determining an alertness inference based on the interaction data, wherein the alertness inference is indicative of a degree of alertness of the user; receiving a second message intended for presentation by the computing device; and altering presentation of the second message on the computing device based on the determined alertness inference, wherein altering the presentation of the second message comprises withholding the presentation of the second message or presenting the second message using a second presentation scheme.

In some cases, presenting the first message using the first presentation scheme comprises presenting the first message with an audible alert, and wherein presenting the second message using the second presentation scheme comprises presenting the second message without an audible alert. In some cases, the interaction data is collected by the computing device. In some cases, the interaction data comprises one or more of biometric data of the individual, inertia data of the computing device, and software-usage data of the computing device. In some cases, the interaction data comprises biometric data of the individual, and wherein the biometric data comprises one or more of eye focus data, blink rate data, and head sway data. In some cases, the interaction data comprises biometric data of the individual, wherein the biometric data comprises biomotion data, and wherein the biomotion data comprises torso movement, limb movement, respiration, head movement, eye movement, hand/finger movement, or cardiac movement. In some cases, the biometric data is collected using a user-facing camera of the computing device. In some cases, the camera could be infrared and/or thermal. In some cases, the biometric data is collected using radiofrequency sensor, such as a continuous wave (CW), pulsed CW, frequency modulated CW (FMCW), ultra-wideband (UWB) or other. In some cases, an UWB sensor may be used for precision location detection, and relative measures to another UWB equipped device such as a smartphone or smart tag. In some cases, the biometric data could be collected via one or more sensors on a device interfaced (such as via a wireless link) to a smartphone, such as a patch or watch. In some cases, the biometric data could be collected on a respiratory therapy (e.g., positive airway pressure (PAP)) device, using sensors such as pressure, flow, and/or sound sensors. In some cases, the interaction data further comprises inertia data of the computing device or software-usage data of the computing device, and wherein determining an alertness inference based on the interaction data comprises determining an alertness inference based on one of the biometric data, the inertia data, and the software-usage data; and confirming the alertness inference using another of the biometric data, the inertia data, and the software-usage data. In some cases, the interaction data comprises software-usage data, and wherein determining the alertness inference comprises generating an alertness score of the user based on at least one of a speed of interaction of the user and an accuracy of interaction of the user.

In some cases, presenting the first message comprises applying a notification rule of the computing device to the first message when received, and wherein altering presentation of the second message comprises modifying the notification rule of the computing device. In some cases, the method further comprises analyzing the second message to determine that the second message is non-essential, wherein altering presentation of the second message is based on the determined alertness inference and the determination that the second message is non-essential. In some cases the method further comprises receiving supplemental information associated with the user interacting with the computing device, wherein the supplemental information comprises at least one of a time of day, a geolocation, a time zone, power data from the computing device, or an ambient light level; wherein determining the alertness inference is further based on the supplemental information.

In some cases, receiving interaction data comprises receiving first message interaction data associated with the user interaction with the presentation of the first message, the method further comprising determining an importance score associated with the first message based on the first message interaction data, wherein receiving the second message comprises assigning a presumed importance score to the second message based on the importance score associated with the first message, and wherein altering presentation of the second message is further based on the presumed importance score of the second message.

In some cases, the method further comprises receiving subsequent interaction data associated with the user subsequently interacting with the computing device; determining a subsequent alertness inference based on the subsequent interaction data, wherein the subsequent alertness inference is indicative of a subsequent degree of alertness of the user that is different than the degree of alertness of the user; presenting the second message according to the first presentation scheme or a third presentation scheme in response to the subsequent alertness inference. In some cases, the second message comprises advertising content, the method further comprising: determining a receptiveness score based on the alertness inference and the interaction data, wherein the receptiveness score is indicative of receptiveness to advertising content, wherein alerting presentation of the second message comprises withholding presentation of the second message when the receptiveness score is below a threshold score; determining a subsequent receptiveness score based on the subsequent alertness inference and the subsequent interaction data, wherein presenting the second message according to the first presentation scheme in response to the subsequent alertness inference occurs when the subsequent receptiveness score is at or above the threshold score. In some cases, determining a receptiveness score comprises determining an importance score associated with an action being taken by the user on the computing device based on received interaction data, wherein the importance score is indicative of a perceived importance of the action to the user based on the received interaction data. In some cases, the action is associated with a particular app on the computing device, and wherein the importance score associated with the action is an importance score associated with the app.

In some cases, the second message comprises advertising content, the method further comprising selecting a route of presentation based on the alertness inference and the received interaction data, wherein altering presentation of the second message comprises presenting the second message using the second presentation scheme, and wherein the second presentation scheme uses the selected route of presentation. In some cases, the alertness inference and the received interaction data is indicative that the user is not watching the computing device, and wherein the selected route of presentation comprises an audio route of presentation.

In some cases, the method further comprises determining that the user is travelling based on the received interaction data, calendar data, or location data; and presenting a travel alert based on the alertness inference. In some cases, the travel alert comprises a reminder to secure the computing device. In some cases, the alertness inference is indicative that the user has a first level of alertness, the method further comprising: receiving subsequent interaction data associated with the user subsequently interacting with the computing device; determining a subsequent alertness inference based on the subsequent interaction data, wherein the subsequent alertness inference is indicative that the user has a second level of alertness that is lower than the first level of alertness; determining that the computing device has not been secured after the travel alert based on the subsequent interaction data; and presenting a subsequent travel alert based on the subsequent alertness inference and the determination that the computing device has not been secured after the travel alert, wherein the subsequent travel alert comprises an alarm to increase alertness of the user and a subsequent reminder to secure the computing device. In some cases, the method further comprises automatically locking the computing device. In some cases, the method further comprises determining that the user is travelling based on the received interaction data, calendar data, or location data, wherein determining that the user is travelling comprises identifying a presumed destination; determining that the user is asleep based on the alertness inference; and automatically setting an alarm after determining that the user is asleep, wherein the alarm is set to wake the user prior to arrival at the presumed destination.

In some cases, the method further comprises determining an importance score associated with an action being taken by the user on the computing device at the time the second message is received based on the received interaction data and the determined alertness inference; and determining an importance score associated with the second message, wherein altering presentation of the second message is further based on comparing the importance score of the second message with the importance score of the action being taken by the user. In some cases, determining the importance score associated with the second message comprises identifying a source of the second message and applying the importance score associated with the source of the second message, wherein the importance score associated with the source of the second message is based on one or more historical importance scores associated with the source of the second message. In some cases, the method further comprises receiving subsequent interaction data associated with the user interacting with the presentation of the second message; and updating the importance score associated with the source of the second message based on the subsequent interaction data.

In some cases, a respiratory therapy (e.g., PAP) user with an associated app could have tailored/personalized advice delivered when they are at a desired alertness level such as to best act on that advice.

In some cases, the computing device is a mobile device comprising an inertia measurement unit for obtaining inertia data. In some cases, the mobile device may further comprise a user-facing camera for obtaining biometric data.

Embodiments of the present disclosure include a system comprising a control system including one or more processors; and a memory having stored thereon machine readable instructions; wherein the control system is coupled to the memory, and the method(s) disclosed herein is/are implemented when the machine executable instructions in the memory are executed by at least one of the one or more processors of the control system.

Embodiments of the present disclosure include a system for monitoring alertness, the system including a control system having one or more processors configured to implement the method(s) disclosed herein.

Embodiments of the present disclosure include a computer program product comprising instructions which, when executed by a computer, cause the computer to carry out the method(s) disclosed herein. In some cases, the computer program product is a non-transitory computer readable medium.

### BRIEF DESCRIPTION OF THE DRAWINGS

The specification makes reference to the following appended figures, in which use of like reference numerals in different figures is intended to illustrate like or analogous components.

FIG. 1 is a schematic block diagram depicting a system for monitoring and leveraging alertness, according to certain aspects of the present disclosure.

FIG. 2 is a perspective view of a user interacting with a computing device with a high level of alertness, according to certain aspects of the present disclosure.

FIG. 3 is a perspective view of a user interacting with a computing device with a low level of alertness, according to certain aspects of the present disclosure.

FIG. 4 is a perspective view of a user that has fallen asleep after interacting with a computing device, according to certain aspects of the present disclosure.

FIG. 5 is a flowchart depicting a process for monitoring and leveraging alertness, according to certain aspects of the present disclosure.

FIG. 6 is a flowchart depicting a process for controlling presentation of a message based on monitored alertness, according to certain aspects of the present disclosure.

FIG. 7 is a flowchart depicting a process for controlling presentation of a message based on a receptiveness score, according to certain aspects of the present disclosure.

FIG. 8 is a combination timeline and table depicting reaction times for messages and resulting importance scores, according to certain aspects of the present disclosure.

FIG. 9 is a table depicting alertness scores, interaction speed/accuracy scores, and importance scores for various actions on a computing device, according to certain aspects of the present disclosure.

FIG. 10 is a flowchart depicting a process for controlling presentation of a travel alert based on an alertness inference, according to certain aspects of the present disclosure.

### DETAILED DESCRIPTION

Certain aspects and features of the present disclosure relate to monitoring and leveraging alertness of a user interacting with a computing device, such as a mobile device (e.g., a smartphone or tablet or smart glasses). By monitoring interaction data, an alertness inference of the user can be generated. The interaction data can include biometric data of the user (e.g., blink rate, eye focus, and breathing rate), inertia data of the device (e.g., swaying and orientation), and software-usage data of the device (e.g., button press speed and accuracy, app or action being used, and response times). The alertness inference can be a score measuring a degree of alertness of the user, from a deep sleep through fully alert. The alertness inference can be leveraged to automatically alert presentation of a message (e.g., notification) on the device, such as withholding presentation of the message or presenting it in different fashion (e.g., silently).

As used herein, the term message can include a collection of data received by a computing device for presentation by the computing device. In some cases, a message can be a notification, such as a notification of an incoming text message, an alert form an app or other piece of software installed on the computing device, a notification of a photograph or other file being transferred to the computing device, or the like. In some cases, a message can include a file sent to or streamed by the computing device. In some cases, a message can include a media file, such as a photo, a sound file, a song file, a video file, or the like. In some cases, a message can include any data received by a computing device for which the computing device has rules or settings defined for how the message is to be automatically presented to a user. In some cases, a message can be an advertisement or can otherwise contain advertising content. In some cases, a message can be represented by the computing device as a text representation, a graphic, a vibration, a light, a sound, or a communication to a remote device (e.g., a smart speaker or a smart light).

In some cases, the present disclosure can beneficially reduce problematic usage of certain computing devices, such as smartphones. Leveraging an alertness inference for a user can permit a computing device to automatically ignore or present unobtrusively certain notifications that may otherwise distract the user. For example, while falling asleep, a user may otherwise be distracted or kept awake due to ongoing messages and notifications. However, if the alertness inference is indicative that the user is falling asleep, it can be used to withhold presentation of various messages or notifications that may be distracting, and which may be more beneficially presented to user when the user is in a more wakeful state. Additionally, the alertness inference can be used to take supplemental action, such as to remind a user to put down the device or to send a command to a remote device (e.g., a command to a smart light to turn off the lights).

Aspects and features of the present disclosure can be used with any suitable computing device. Examples of suitable computing devices include smartphones, tablets, computers, and the like, although any suitable computing device can be used. In an example, the present disclosure can be especially useful for users who interact with smartphones or tablets at times when they may be falling asleep. In another example, the present disclosure can be especially useful for users who are interacting with computers at times when their alertness may be at maximum or minimum levels. In another example, a user of a virtual reality headset may be able to have the presentation of messages (e.g., notifications) controlled based on the user's alertness level and/or an importance score associated with the messages and the action being taken by the user on the headset. In another example, a user watching a television or playing a console video game may be able to have the presentation of various messages controlled based on the user's alertness level.

While described herein primarily with reference to a computing device, in some cases certain aspects and features of the disclosure can be implemented across multiple computing devices. For example, a cloud-accessible or network-accessible device may be used for certain processing, while a personal device (e.g., smartphone) may be used to control the presentation of messages. In another example, a first device (e.g., smartphone) may control the presentation of its messages based on an alertness inference of a user using a second device (e.g., a television or gaming console).

Interaction data can be used to generate the alertness inference. The interaction data can include data related to the user interacting with the computing device, although that need not always be the case. In some cases, interaction data can include data related to the user interacting with a second device. The interaction data can come entirely from the computing device itself, although that need not always be the case. In some cases, a remote device (e.g., a remote camera or other sensor) can provide some or all interaction data associated with the user interacting with the computing device. In cases where interaction data is supplied from a remote device, it can be supplied to the computing device for processing and/or further action (e.g., altering presentation of a message).

Interaction data can be active or passive. Active interaction data is data collected by one or more computing devices as a user interacts with the one or more computing devices. For example, active interaction data can include data associated with a user interacting with a message presented on the computing device, although that need not always be the case. In some cases, active interaction data includes data associated with a user otherwise interacting with a computing device, such as browsing a website, reading an email, playing a game, adjusting a setting on a respiratory therapy companion app, or the like. Passive interaction data is data collected by one or more computing devices while the user is not directly interacting with the one or more computing devices. For example, passive interaction data can include data collected by the one or more computing devices as the user brushes their teeth, reads a book, exercises, eats, sleeps, sleeps while wearing a respiratory therapy device, or the like. In some cases, passive interaction data includes data collected before, after, and/or between collection of active interaction data.

The interaction data can include biometric data, inertia data, software-usage data, or any combination thereof. In some cases, an alertness inference can be made using one or more of the different types of interaction data, while one or more other types of interaction data are used to confirm or refute the alertness inference. For example, biometric data suggesting low alertness may be used to generate an alertness inference that the user is falling asleep, however such an inference may be refuted by software-usage data clearly indicating that the user is pressing buttons (e.g., on-screen buttons) rapidly and with a high degree of accuracy, suggesting that the user may have a higher level of alertness.

Biometric data includes data about the user's biological traits as the user interacts with the device. Examples of such biological traits include blink rate, eye movement, eye focus (e.g., direction of focus), heart rate, breathing rate, head movement, and lip movement. Other biological traits can be used as well. Biometric data can include data associated with any combination of biological traits, as well as data derived from such data. Various measurements can be obtained for various biological traits. In some cases, measurements of various biological traits can be used to generate scores for certain alertness indicators, which can be used as inputs for an alertness inference generator. For example, measurements of the user's blink rate can be taken over time and used to generate a blink score, which can be used as an input to a system generating an alertness inference. In some cases, however, raw and/or pre-processed biometric data can be used to generate an alertness inference. In some cases, biometric data can include biomotion data. Biomotion data can include any detectable motion of a user or a body part of a user, such as torso movement, limb movement, respiration, or cardiac movement.

Inertia data includes data associated with the computing device's movement and/or position in space. Inertia data can include any data originating from a computing device's inertial measurement unit (IMU), or any similar sensors (e.g., accelerometers, gyroscopes, and the like). The IMU or similar sensor can be solid state, although that need not always be the case. Examples of suitable inertia data can include 3D acceleration data, orientation data, specific force data, angular rate data, and any combination thereof, as well as data derived from such data. The inertia data can be used to determine how the user is holding or supporting the device, as well as to determine where the device may be located. For example, inertia data consistent with the user slowly swaying or slowly bobbing the device may be indicative that the user is falling asleep, whereas inertia data showing the device is being held steadily in a hand may be indicative that the user is alert. In some cases, inertia data can be indicative that the device is laying down on a user or on another surface, which may be indicative that the user is not alert. Other inferences can be made using the inertia data.

Software-usage data can include any data associated with software being run on the computing device as the user interacts with the device. Generally, software-usage data is associated with the user interacting with software on the device, such as data indicative of a user pressing buttons in the software or typing in the software, although that need not always be the case. As used herein, the term button in inclusive of a physical button on a computing device or a virtual button, such as a location on a display where the user can press to interact with the software. Buttons can be visual (e.g., in the shape of a button, an icon, or any other visual indicator) or invisible (e.g., a region on the screen where the user may tap to interact regardless of any underlying visual display). In some cases, a button can refer to a specific visual or non-visual target on a display. Examples of suitable software-usage data include button press speed, button press accuracy (e.g., distance or average distance from center of a button to the user's point of touching the button), reaction time to audio and/or visual stimulus (e.g., reaction time to receiving a notification), information about the current app being used, information about the current action being taken by the user, and any combination thereof, including data derived from such data. Software-usage data can be indicative of an alertness level of a user. For example, low button press accuracy or long reaction times to audio and/or visual stimulus may be indicative that the user has a low alertness level.

As described herein, an alertness inference can be leveraged to perform various actions. Examples of suitable actions include sending commands to remote devices, providing alerts (e.g., visual, audio, or haptic alerts), sending commands to software running on the device, and performing automatic actions on the device (e.g., to lock the device). In some cases, the alertness inference can be used to alter a presentation of a message, which can include withholding presentation of the message or presenting a message in a fashion different than how the message would otherwise be presented. For example, while an incoming text message may normally be presented using a visual indicator (e.g., a drop-down message) and an accompanying audio indicator (e.g., a chime or tone), an alertness inference can be leveraged to change how a new incoming text message is presented, such as by presenting the text message without an audio indicator. In some cases, the alertness inference can be used to modify a rule (e.g., a notification rule) for presenting messages on the computing device. Modifying the rule can include modifying the rule for a preset duration (e.g., a set number of hours or until a set time) or as long as the alertness inference remains above or below a particular threshold.

Certain aspects and features of the present disclosure further relate to applying an alertness inference to determine an importance score of an app or action being used by the user. For example, high average alertness scores for a user while interacting with a first app may be indicative that the app is important to the user, whereas low average alertness scores for a user while the user interacts with a second app may be indicative that the second app is less important to the user. Additionally, interaction data can be used with or without an alertness inference to determine an importance score associated with other aspects of the user's interaction with the computing devices, such as an importance score associated with incoming messages. In an example, an incoming message can be associated with a source (e.g., an app, a service, or an individual) which can have an importance score. The importance score of the source can be updated as the user interacts with the source or messages from the source. For example, if a user typically (e.g. at or more than a threshold frequency, such as 7 times out of 10) responds quickly to text messages from a particular individual, that individual may have a relatively high importance score. However, if a user typically hides or ignores notifications from a particular app, that app may receive a relatively low importance score.

The importance score can be used to determine whether or not to alter presentation of a received message, such as to withhold the message or present it in a different fashion. In some cases, when an app with a particular importance score is being used, messages having an importance score below that of the app may be withheld of presented differently, whereas messages having an importance score at or above that of the app may be presented as usual. In some cases, a buffer of a certain number of points can be used to define a threshold above which a message would be presented as usual. For example, a buffer of 20 points could allow a message associated with an importance score of 64 to be presented despite the user working in an app having an importance score of 70. In some cases, this controlled presentation of messages based on importance score only occurs when the user interacting with the app has an alertness inference that is above a threshold, indicative that the user is actively engaging with the app in question. Thus, if the alertness inference is low, it may be inferred that the user is not engaging with the app in question to a degree that presentation of a message with a lower importance score would be problematic. In some cases, multiple messages from the same source (e.g., an app or an individual) within a certain timeframe may be indicative of an increased importance, and therefore may be attributed temporarily increased importance scores based on the number of messages received in the timeframe.

As used herein, altering presentation of a message is inclusive of withholding presentation of the message or presenting the message in a different fashion than if alteration of the presentation of the message had not occurred. When a message is withheld, the message can be presented at a later time. A message for which presentation was previously withheld can be presented after a set duration of time (e.g., a number of minutes, hours, days, or the like), after a set time (e.g., after 6:00 AM the next day), when a subsequent alertness inference for the user changes (e.g., an indication that the user is now more alert, such as after waking the next day), or after any other suitable trigger. In some cases, a message that is withheld can automatically attempt to be re-delivered occasionally (e.g., every 10 minutes, 30 minutes, 60 minutes, or other appropriate time period) or after a set time (e.g., after 6:00 AM the next day). Such re-delivery attempts can be handled as if the message were received anew, with aspects of the present disclosure altering presentation of the message (e.g., further withholding the message) again. In some cases, a message can be automatically presented (e.g., presented in a normal fashion or presented in an altered fashion without being withheld) after it has undergone a threshold number of redelivery attempts. In some cases, a re-delivery attempt can include temporarily increasing an importance score associated with a message based on the number of previous delivery attempts for that message, such that a message throttled by importance scores would nevertheless be presented after a certain number of re-delivery attempts.

In some cases, an alertness inference can further be used to perform travel-related actions when it is determined that the user is travelling. For example, if the user is travelling, an alertness score below a threshold may trigger a travel alert to be presented, such as to awaken the user or remind he user to stow or secure the computing device. In some cases, if no action is taken the by user after a predetermined duration of time or after a subsequent alertness inference indicates the user is less alert or below a certain threshold of alertness (e.g., asleep), a subsequent action may be taken, such as to automatically lock the computing device or to present an alarm to awaken the user to remind the user to stow or secure the computing device. Other travel-related actions can be taken based on an alertness inference, such as to automatically set a time-based or location-based alarm to alert the user prior to arriving at a destination, automatically locking a device or arming an alarm on a device, or presenting useful warnings or reminders to a user (e.g., safety warnings or reminders related to travel). As used herein, travel includes forms of travel where the user is a passenger in a vehicle (e.g., bus, train, car, airplane, helicopter, and the like). In some cases, the computing device can determine that the user is travelling by accessing travel-related information accessible to the computing device, such as a travel itinerary, calendar data, ticket data, or the like. In some cases, the computing device can determine that the user is travelling by analyzing location data or any of the received interaction data to make an inference that the user is travelling. In some cases, such as when an alarm is set to awaken or alert the user before arriving at a destination, the destination information can be obtained from historical interaction data (e.g., analyzing historical interaction data to identify when the user has reached the destination in the past and using that information to infer when the user will reach the destination on the current trip) or from supplemental information (e.g., calendar data, location data, ticket data, and the like).

In some cases, travel can be determined based on sensor data from the computing device, such as RF data associated with radio beacons (e.g., Bluetooth beacons), connections to or presence of other radio devices (e.g., WiFi hotspots and/or cell towers).

In some cases, certain aspects and features of the present disclosure can be used with a respiratory therapy device. The respiratory therapy device can provide pressurized air to a user's respiratory system via a conduit coupled to the user's respiratory system via a user interface. The respiratory therapy device can include a computing device (e.g., a control system) and/or can interact with a computing device (e.g., a user device, such as a smartphone). Certain aspects and features of the present disclosure can include generating an alertness inference based on how a user interacts with a computing device associated with the respiratory therapy system. For example, an alertness inference can be generated using i) biometric data collected by the respiratory therapy device; ii) the user interacting with an interface display screen on the respiratory therapy device; iii) sensor data collected by the respiratory therapy device (e.g., detection of user interface leaks); iv) interaction with a companion app on a user device (e.g., smartphone) that communicates with the respiratory therapy device; v) any combination of i-iv. The alertness inference can be generated using other data associated with the user's interaction with a computing device associated with a respiratory therapy device.

In some cases, an alertness inference can be used to alter presentation of a message by a computer associated with the respiratory therapy device. For example, a message that is to be displayed by a computing device of the respiratory therapy device or by a computing device associated with the respiratory therapy device can be altered as described herein, based on the alertness inference. In an example, presentation of certain respiratory therapy-related messages (e.g., therapy information, leak alerts, co-morbidity information, user interface resupply information, and the like) can be altered based on the alertness inference.

Use of certain aspects of the present disclosure can improve the efficacy of respiratory therapy and sleep-related therapy. For example, by detecting an alertness inference, a computing device associated with a respiratory therapy device can delay or otherwise alter presentation of a message or alert when it is determined that the user is falling asleep or has fallen asleep, thereby not engaging or awakening the user unnecessarily. A respiratory therapy device enhanced with such aspects of the present disclosure can be beneficial over respiratory therapy device without such enhancements, and can improve the respiratory therapy device's ability to treat sleep-related and/or respiratory disorders.

Many individuals suffer from sleep-related and/or respiratory disorders. Examples of sleep-related and/or respiratory disorders include Periodic Limb Movement Disorder (PLMD), Restless Leg Syndrome (RLS), Sleep-Disordered Breathing (SDB) such as Obstructive Sleep Apnea (OSA), Central Sleep Apnea (CSA), and other types of apneas such as mixed apneas and hypopneas, Respiratory Effort Related Arousal (RERA), Cheyne-Stokes Respiration (CSR), respiratory insufficiency, Obesity Hyperventilation Syndrome (OHS), Chronic Obstructive Pulmonary Disease (COPD), Neuromuscular Disease (NMD), and chest wall disorders.

Obstructive Sleep Apnea (OSA) is a form of Sleep Disordered Breathing (SDB), and is characterized by events including occlusion or obstruction of the upper air passage during sleep resulting from a combination of an abnormally small upper airway and the normal loss of muscle tone in the region of the tongue, soft palate and posterior oropharyngeal wall. More generally, an apnea generally refers to the cessation of breathing caused by blockage of the air (Obstructive Sleep Apnea) or the stopping of the breathing function (often referred to as Central Sleep Apnea). Typically, the individual will stop breathing for between about 15 seconds and about 30 seconds during an obstructive sleep apnea event.

Other types of apneas include hypopnea, hyperpnea, and hypercapnia. Hypopnea is generally characterized by slow or shallow breathing caused by a narrowed airway, as opposed to a blocked airway. Hyperpnea is generally characterized by an increase depth and/or rate of breathing. Hypercapnia is generally characterized by elevated or excessive carbon dioxide in the bloodstream, typically caused by inadequate respiration.

Cheyne-Stokes Respiration (CSR) is another form of sleep disordered breathing. CSR is a disorder of a patient's respiratory controller in which there are rhythmic alternating periods of waxing and waning ventilation known as CSR cycles. CSR is characterized by repetitive de-oxygenation and re-oxygenation of the arterial blood.

Obesity Hyperventilation Syndrome (OHS) is defined as the combination of severe obesity and awake chronic hypercapnia, in the absence of other known causes for hypoventilation. Symptoms include dyspnea, morning headache and excessive daytime sleepiness.

Chronic Obstructive Pulmonary Disease (COPD) encompasses any of a group of lower airway diseases that have certain characteristics in common, such as increased resistance to air movement, extended expiratory phase of respiration, and loss of the normal elasticity of the lung.

Neuromuscular Disease (NMD) encompasses many diseases and ailments that impair the functioning of the muscles either directly via intrinsic muscle pathology, or indirectly via nerve pathology. Chest wall disorders are a group of thoracic deformities that result in inefficient coupling between the respiratory muscles and the thoracic cage.

A Respiratory Effort Related Arousal (RERA) event is typically characterized by an increased respiratory effort for ten seconds or longer leading to arousal from sleep and which does not fulfill the criteria for an apnea or hypopnea event. RERAs are defined as a sequence of breaths characterized by increasing respiratory effort leading to an arousal from sleep, but which does not meet criteria for an apnea or hypopnea. These events must fulfil both of the following criteria: (1) a pattern of progressively more negative esophageal pressure, terminated by a sudden change in pressure to a less negative level and an arousal, and (2) the event lasts ten seconds or longer. In some implementations, a Nasal Cannula/Pressure Transducer System is adequate and reliable in the detection of RERAs. A RERA detector may be based on a real flow signal derived from a respiratory therapy device. For example, a flow limitation measure may be determined based on a flow signal. A measure of arousal may then be derived as a function of the flow limitation measure and a measure of sudden increase in ventilation. One such method is described in WO 2008/138040, assigned to ResMed Ltd., the disclosure of which is hereby incorporated by reference herein in its entirety.

These and other disorders are characterized by particular events (e.g., snoring, an apnea, a hypopnea, a restless leg, a sleeping disorder, choking, an increased heart rate, labored breathing, an asthma attack, an epileptic episode, a seizure, or any combination thereof) that occur when the individual is sleeping.

The Apnea-Hypopnea Index (AHI) is an index used to indicate the severity of sleep apnea during a sleep session. The AHI is calculated by dividing the number of apnea and/or hypopnea events experienced by the user during the sleep session by the total number of hours of sleep in the sleep session. The event can be, for example, a pause in breathing that lasts for at least 10 seconds. An AHI that is less than 5 is considered normal. An AHI that is greater than or equal to 5, but less than 15 is considered indicative of mild sleep apnea. An AHI that is greater than or equal to 15, but less than 30 is considered indicative of moderate sleep apnea. An AHI that is greater than or equal to 30 is considered indicative of severe sleep apnea. In children, an AHI that is greater than 1 is considered abnormal. Sleep apnea can be considered “controlled” when the AHI is normal, or when the AHI is normal or mild. The AHI can also be used in combination with oxygen desaturation levels to indicate the severity of Obstructive Sleep Apnea.

Many individuals suffer from insomnia, a condition which is generally characterized by a dissatisfaction with sleep quality or duration (e.g., difficulty initiating sleep, frequent or prolonged awakenings after initially falling asleep, and an early awakening with an inability to return to sleep). It is estimated that over 2.6 billion people worldwide experience some form of insomnia, and over 750 million people worldwide suffer from a diagnosed insomnia disorder. In the United States, insomnia causes an estimated gross economic burden of $107.5 billion per year, and accounts for 13.6% of all days out of role and 4.6% of injuries requiring medical attention. Recent research also shows that insomnia is the second most prevalent mental disorder, and that insomnia is a primary risk factor for depression.

Nocturnal insomnia symptoms generally include, for example, reduced sleep quality, reduced sleep duration, sleep-onset insomnia, sleep-maintenance insomnia, late insomnia, mixed insomnia, and/or paradoxical insomnia. Sleep-onset insomnia is characterized by difficulty initiating sleep at bedtime. Sleep-maintenance insomnia is characterized by frequent and/or prolonged awakenings during the night after initially falling asleep. Late insomnia is characterized by an early morning awakening (e.g., prior to a target or desired wakeup time) with the inability to go back to sleep. Comorbid insomnia refers to a type of insomnia where the insomnia symptoms are caused at least in part by a symptom or complication of another physical or mental condition (e.g., anxiety, depression, medical conditions, and/or medication usage). Mixed insomnia refers to a combination of attributes of other types of insomnia (e.g., a combination of sleep-onset, sleep-maintenance, and late insomnia symptoms). Paradoxical insomnia refers to a disconnect or disparity between the user's perceived sleep quality and the user's actual sleep quality.

Diurnal (e.g., daytime) insomnia symptoms include, for example, fatigue, reduced energy, impaired cognition (e.g., attention, concentration, and/or memory), difficulty functioning in academic or occupational settings, and/or mood disturbances. These symptoms can lead to psychological complications such as, for example, lower performance, decreased reaction time, increased risk of depression, and/or increased risk of anxiety disorders. Insomnia symptoms can also lead to physiological complications such as, for example, poor immune system function, high blood pressure, increased risk of heart disease, increased risk of diabetes, weight gain, and/or obesity.

Co-morbid Insomnia and Sleep Apnea (COMISA) refers to a type of insomnia where the subject experiences both insomnia and obstructive sleep apnea (OSA). OSA can be measured based on an Apnea-Hypopnea Index (AHI) and/or oxygen desaturation levels. The AHI is calculated by dividing the number of apnea and/or hypopnea events experienced by the user during the sleep session by the total number of hours of sleep in the sleep session. The event can be, for example, a pause in breathing that lasts for at least 10 seconds. An AHI that is less than 5 is considered normal. An AHI that is greater than or equal to 5, but less than 15 is considered indicative of mild OSA. An AHI that is greater than or equal to 15, but less than 30 is considered indicative of moderate OSA. An AHI that is greater than or equal to 30 is considered indicative of severe OSA. In children, an AHI that is greater than 1 is considered abnormal.

Insomnia can also be categorized based on its duration. For example, insomnia symptoms are considered acute or transient if they occur for less than 3 months. Conversely, insomnia symptoms are considered chronic or persistent if they occur for 3 months or more, for example. Persistent/chronic insomnia symptoms often require a different treatment path than acute/transient insomnia symptoms.

Known risk factors for insomnia include gender (e.g., insomnia is more common in females than males), family history, and stress exposure (e.g., severe and chronic life events). Age is a potential risk factor for insomnia. For example, sleep-onset insomnia is more common in young adults, while sleep-maintenance insomnia is more common in middle-aged and older adults. Other potential risk factors for insomnia include race, geography (e.g., living in geographic areas with longer winters), altitude, and/or other sociodemographic factors (e.g. socioeconomic status, employment, educational attainment, self-rated health, etc.).

Mechanisms of insomnia include predisposing factors, precipitating factors, and perpetuating factors. Predisposing factors include hyperarousal, which is characterized by increased physiological arousal during sleep and wakefulness. Measures of hyperarousal include, for example, increased levels of cortisol, increased activity of the autonomic nervous system (e.g., as indicated by increase resting heart rate and/or altered heart rate), increased brain activity (e.g., increased EEG frequencies during sleep and/or increased number of arousals during REM sleep), increased metabolic rate, increased body temperature and/or increased activity in the pituitary-adrenal axis. Precipitating factors include stressful life events (e.g., related to employment or education, relationships, etc.) Perpetuating factors include excessive worrying about sleep loss and the resulting consequences, which may maintain insomnia symptoms even after the precipitating factor has been removed.

Once diagnosed, insomnia can be managed or treated using a variety of techniques or providing recommendations to the patient. Generally, the patient can be encouraged or recommended to generally practice healthy sleep habits (e.g., plenty of exercise and daytime activity, have a routine, no bed during the day, eat dinner early, relax before bedtime, avoid caffeine in the afternoon, avoid alcohol, make bedroom comfortable, remove bedroom distractions, get out of bed if not sleepy, try to wake up at the same time each day regardless of bed time) or discouraged from certain habits (e.g., do not work in bed, do not go to bed too early, do not go to bed if not tired). The patient can additionally or alternatively be treated using sleep medicine and medical therapy such as prescription sleep aids, over-the-counter sleep aids, and/or at-home herbal remedies.

The patient can also be treated using cognitive behavior therapy (CBT) or cognitive behavior therapy for insomnia (CBT-I), which generally includes sleep hygiene education, relaxation therapy, stimulus control, sleep restriction, and sleep management tools and devices. Sleep restriction is a method designed to limit time in bed (the sleep window or duration) to actual sleep, strengthening the homeostatic sleep drive. The sleep window can be gradually increased over a period of days or weeks until the patient achieves an optimal sleep duration. Stimulus control includes providing the patient a set of instructions designed to reinforce the association between the bed and bedroom with sleep and to reestablish a consistent sleep-wake schedule (e.g., go to bed only when sleepy, get out of bed when unable to sleep, use the bed for sleep only (e.g., no reading or watching TV), wake up at the same time each morning, no napping, etc.) Relaxation training includes clinical procedures aimed at reducing autonomic arousal, muscle tension, and intrusive thoughts that interfere with sleep (e.g., using progressive muscle relaxation). Cognitive therapy is a psychological approach designed to reduce excessive worrying about sleep and reframe unhelpful beliefs about insomnia and its daytime consequences (e.g., using Socratic question, behavioral experiences, and paradoxical intention techniques). Sleep hygiene education includes general guidelines about health practices (e.g., diet, exercise, substance use) and environmental factors (e.g., light, noise, excessive temperature) that may interfere with sleep. Mindfulness-based interventions can include, for example, meditation.

Certain aspects of the present disclosure can facilitate promoting healthy sleep habits and facilitate the efficacy of sleep aids or sleep-related therapy, such as by withholding or otherwise altering the presentation of messages based on an alertness inference. For example, when an alertness inference is indicative that the user is starting to fall asleep, messages that may inhibit or negatively impact a user's ability to fall asleep (e.g., loud messages, bright messages, messages with strong or alarming content, messages that prompt strong user interaction, highly stimulating messages, and the like) can be withheld or otherwise altered to reduce any negative impact they might have on the user's attempt to fall asleep.

These illustrative examples are given to introduce the reader to the general subject matter discussed here and are not intended to limit the scope of the disclosed concepts. The following sections describe various additional features and examples with reference to the drawings in which like numerals indicate like elements, and directional descriptions are used to describe the illustrative embodiments but, like the illustrative embodiments, should not be used to limit the present disclosure. The elements included in the illustrations herein may not be drawn to scale.

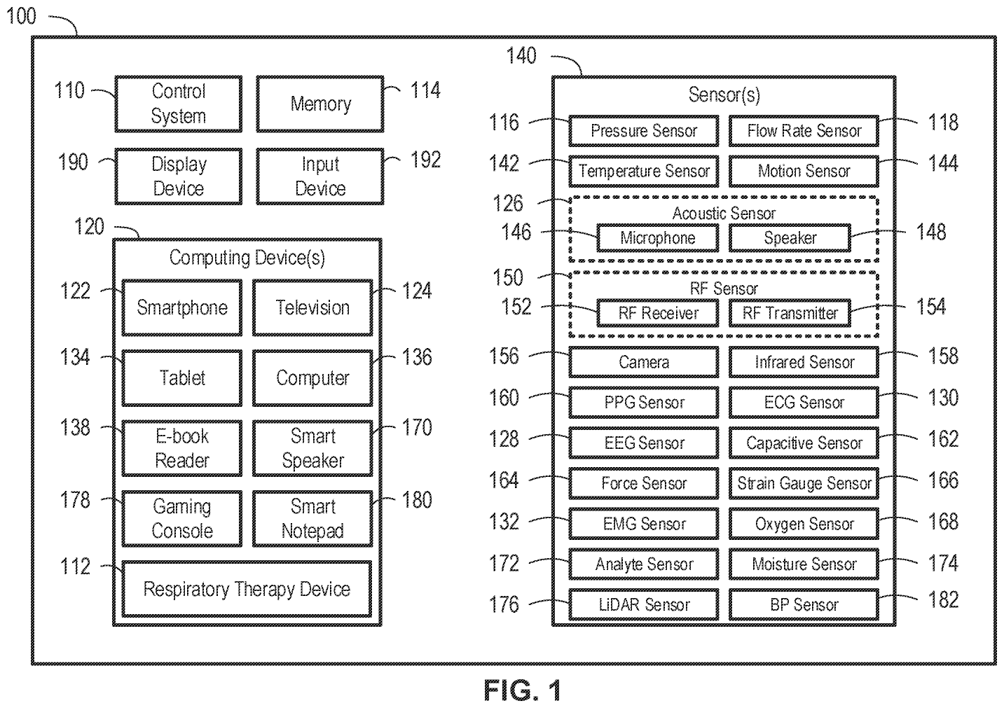

FIG. 1 is a schematic block diagram depicting a system 100 for monitoring and leveraging alertness, according to certain aspects of the present disclosure. The system 100 includes a control system 110, a memory device 114, one or more computing devices 120, and one or more sensors 140. In some cases, the system 100 also includes a display device 190 and an input device 192. The system 100 generally can be used to collect and/or generate interaction data associated with a user (e.g., an individual, a person, or the like) interacting with the system 100, such as with the one or more electronic devices 120. The system 100 can use the interaction data to generate an alertness inference, such as an alertness score (e.g., on a numerical scale) or an alertness classification (e.g., an enumerated state such as “asleep,” “dozing off,” “low alertness” or “drowsy,” “medium alertness” or “wakefulness,” “fully alert” or “awake,” or “hyper-alert”). The system 100 can make use of data from the one or more sensors 140 to collect certain interaction data, such as biometric data associated with the user interacting with the one or more computing devices 120. For example, the system 100 can receive interaction data that is biometric data in the form of a number of eye blinks per minute by leveraging sensor data from a camera 156, an infrared sensor 158, and/or an RF sensor 150. Such sensor data can be analyzed, optionally with other interaction data, by the system 100 (e.g., using one or more trained algorithms) to generate the alertness inference, which can in turn be used to alter presentation of a received message.

The system 100 can receive messages, such as via the one or more computing devices 120. Messages can originate external to or internal to the system 100. An internal message may originate from an app or other software running on the one or more computing devices 120. An external message may originate from an individual (e.g., a text message) and/or via software running on a remote device (e.g., a push notification from a remote server, or a communication from another mobile or fixed device).

The control system 110 includes one or more processors. As such, the control system 110 can include any suitable number of processors (e.g., one processor, two processors, five processors, ten processors, and the like). In some cases, the control system 110 includes one or more processors, one or more memory devices (e.g., the memory device 114, or a different memory device), one or more other electronic components (e.g., one or more electronic chips and/or components, one or more printed circuit boards, one or more power units, one or more graphical processing units, one or more input devices, one or more output devices, one or more secondary storage devices, one or more primary storage devices, and the like), or any combination thereof. In some implementations, the control system 110 includes the memory device 114 or a different memory device, yet in other implementations, the memory device 114 is separate and distinct from the control system 110, but in communication with the control system 110.

The control system 110 generally controls (e.g., drives) the various components of the system 100 and/or analyzes data obtained and/or generated by the components of the system 100. For example, the control system 110 is arranged to receive sensor data from the one or more sensors 140 and provide control signals to the one or more computing devices 120. The control system 110 executes machine readable instructions that are stored in the memory device 114 or a different memory device. The one or more processors of the control system 110 can be general or special purpose processors and/or microprocessors.

While the control system 110 is described and depicted in FIG. 1 as being a separate and distinct component of the system 100, in some implementations, the control system 110 is integrated in and/or directly coupled to the one or more computing devices 120 and/or the one or more sensors 140. The control system 110 can be coupled to and/or positioned within a housing of the one or more computing devices 120, one or more of the sensors 140, or any combination thereof. The control system 110 can be centralized (within one housing) or decentralized (within two or more physically distinct housings). Likewise, the one or more sensors 140 can be integrated in and/or directly coupled to the one or more computing devices 120, and can be coupled to and/or positioned within a housing of the one or more computing devices 120. For example, in some cases, system 100 can be embodied in a single housing of a mobile device, such as a smartphone or tablet. Such mobile device can be a smartphone 122 or tablet 134 and can include the control system 110 (e.g., via one or more processors of the mobile device), memory 114 (e.g., via internal memory and storage), display device 190 and input device 192 (e.g., via a touchscreen), and the one or more sensors 140 (e.g., via cameras, inertial measurement units, and other components of the device). In some cases, one or more of the one or more computing devices 120 can be a fixed device on a mobile platform (e.g., an infotainment system of a car, airplane, or bus; a computing device of a vehicle; and the like). In some cases, a mobile device could be a watch or a tag/tracker tile.

While the system 100 is shown as including a single memory device 114, it is contemplated that the system 100 can include any suitable number of memory devices (e.g., one memory device, two memory devices, five memory devices, ten memory devices, and the like). The memory device 114 can be any suitable computer readable storage device or media, such as, for example, a random or serial access memory device, a hard drive, a solid state drive, a flash memory device, and the like. The memory device 114 can be coupled to and/or positioned within a housing of the one or more computing devices 120, the one or more of the sensors 140, the control system 110, or any combination thereof. The memory device 114 can be centralized (within one housing) or decentralized (within two or more physically distinct housings).

The one or more computing devices 120 can include a smartphone 122, a television 124 (e.g., a smart television), a tablet 134, a computer 136, an e-book reader 138, a smart speaker 170, a gaming console 178, a smart notepad 180, a respiratory therapy device 112, or any combination thereof. Other computing devices can be used. In some cases, the one or more computing devices 120 is a mobile device. In some cases, the one or more computing devices 120 is a portable device comprising a portable power source, such as a battery. In some cases, the one or more computing devices 120 include a network interface for communicating with a network, such as a local area network, a personal area network, an intranet, the Internet, or a cloud network. In some cases, the one or more computing devices 120 can include a network interface for receiving messages from a remote source.

When the computing device(s) 120 includes a respiratory therapy device 112, the respiratory therapy device 112 can include any suitable device for providing respiratory therapy, optionally including corresponding components. For example, a respiratory therapy device 112 can include a control system (e.g., control system 110), a flow generator, a user interface, a conduit (also referred to as a tube or an air circuit), a display device (e.g., display device 190), a humidifier, and the like. Respiratory pressure therapy refers to the application of a supply of air to an entrance to a user's airways at a controlled target pressure that is nominally positive with respect to atmosphere throughout the user's breathing cycle (e.g., in contrast to negative pressure therapies such as the tank ventilator or cuirass). The respiratory therapy device 112 is generally used to treat individuals suffering from one or more sleep-related respiratory disorders (e.g., obstructive sleep apnea, central sleep apnea, or mixed sleep apnea). The respiratory therapy device 112 generally aids in increasing the air pressure in the throat of a user to aid in preventing the airway from closing and/or narrowing during sleep.

The respiratory therapy device 112 can be used, for example, as a ventilator or as a positive airway pressure (PAP) system, such as a continuous positive airway pressure (CPAP) system, an automatic positive airway pressure system (APAP), a bi-level or variable positive airway pressure system (BPAP or VPAP), or any combination thereof. The CPAP system delivers a predetermined air pressure (e.g., determined by a sleep physician) to the user. The APAP system automatically varies the air pressure delivered to the user based on, for example, respiration data associated with the user. The BPAP or VPAP system is configured to deliver a first predetermined pressure (e.g., an inspiratory positive airway pressure or IPAP) and a second predetermined pressure (e.g., an expiratory positive airway pressure or EPAP) that is lower than the first predetermined pressure.

The respiratory therapy device 112 can include a housing, a blower motor, an air inlet, and an air outlet. The blower motor is at least partially disposed or integrated within the housing. The blower motor draws air from outside the housing (e.g., atmosphere) via the air inlet and causes pressurized air to flow through the humidifier, and through the air outlet. In some implementations, the air inlet and/or the air outlet include a cover that is moveable between a closed position and an open position (e.g., to prevent or inhibit air from flowing through the air inlet or the air outlet).

The user interface engages a portion of the user's face and delivers pressurized air from the respiratory therapy device 112 to the user's airway to aid in preventing the airway from narrowing and/or collapsing during sleep. This may also increase the user's oxygen intake during sleep. Generally, the user interface engages the user's face such that the pressurized air is delivered to the user's airway via the user's mouth, the user's nose, or both the user's mouth and nose. Together, the respiratory therapy device 112, the user interface, and the conduit form an air pathway fluidly coupled with an airway of the user. The pressurized air also increases the user's oxygen intake during sleep. Depending upon the therapy to be applied, the user interface may form a seal, for example, with a region or portion of the user's face, to facilitate the delivery of gas at a pressure at sufficient variance with ambient pressure to effect therapy, for example, at a positive pressure of about 10 cmH₂O relative to ambient pressure. For other forms of therapy, such as the delivery of oxygen, the user interface may not include a seal sufficient to facilitate delivery to the airways of a supply of gas at a positive pressure of about 10 cmH₂O.

While the respiratory therapy device 112 has been described herein as including each of the flow generator, the user interface, the conduit, the display device, and the humidifier, more or fewer components can be included in a respiratory therapy system according to implementations of the present disclosure. The respiratory therapy device 112, and any associated components (e.g., the user interface, the conduit, the display device, and the humidification tank) can contain one or more sensors (e.g., a pressure sensor, a flow rate sensor, or more generally any of the other sensors 140 described herein). These one or more sensors can be used, for example, to measure the air pressure and/or flow rate of pressurized air supplied by the respiratory therapy device 112.

While the one or more computing devices 120 are shown and described as including the smartphone 122, the television 124, the tablet 134, the computer 136, the e-book reader 138, the smart speaker 170, the gaming console 178, the smart notepad 180, and the respiratory therapy device 112, more generally, the one or more computing devices 120 of the system 100 can include any combination and/or any number of the computing devices described and/or shown herein, as well as other suitable computing devices. For example, in some cases, the one or more computing devices 120 of the system 100 only include the smartphone 122. For another example, in some cases, the one or more computing devices 120 of the system 100 only include the smartphone 122 and tablet 134. In another example, the one or more computing devices 120 can include a smartphone 122 and a respiratory therapy device 112. Various other combinations and/or numbers of the one or more computing devices 120 are contemplated.

The one or more sensors 140 include a pressure sensor 116, a flow rate sensor 118, temperature sensor 142, a motion sensor 144, an acoustic sensor 126 (e.g., a microphone 146 and/or a speaker 148), a radio-frequency (RF) sensor 150 (e.g., an RF receiver 152 and/or an RF transmitter 154), a camera 156, an infrared sensor 158, a photoplethysmogram (PPG) sensor 160, an electrocardiogram (ECG) sensor 130, an electroencephalography (EEG) sensor 128, a capacitive sensor 162, a force sensor 164, a strain gauge sensor 166, an electromyography (EMG) sensor 132, an oxygen sensor 168, an analyte sensor 178, a moisture sensor 174, a LiDAR sensor 178, or any combination thereof, as well as other suitable sensors. Generally, each of the one or more sensors 140 is configured to output sensor data that can be received and/or stored in the memory device 114 or one or more different memory devices. The sensor data can be analyzed by the control system 110 for use in determining an alertness inference. In some cases, the sensor data can also be used to calibrate one or more of the one or more sensors 140 and/or to train a machine learning algorithm. In some cases, an algorithm can be trained based on sensor data (e.g., physiological or biometric data derived from sensor data) and corresponding alertness associated with a given user or a population of users.

While the one or more sensors 140 are shown and described as including the pressure sensor 116, flow rate sensor 118, temperature sensor 142, the motion sensor 144, acoustic sensor 126 (e.g., the microphone 146 and/or the speaker 148), the RF sensor 150 (e.g., the RF receiver 152 and/or the RF transmitter 154), the camera 156, the infrared sensor 158, the PPG sensor 160, the ECG sensor 130, the EEG sensor 128, the capacitive sensor 162, the force sensor 164, the strain gauge sensor 166, the EMG sensor 132, the oxygen sensor 168, the analyte sensor 178, the moisture sensor 174, and the LiDAR sensor 178, more generally, the one or more sensors 140 of the system 100 can include any combination and/or any number of the sensors 140 described and/or shown herein, as well as other suitable sensors. In one example, the one or more sensors 140 of the system 100 only include the camera 156. In another example, the one or more sensors 140 of the system 100 only include the microphone 146 and the speaker 148. Various other combinations and/or numbers of the one or more sensors 140 are contemplated. In some cases, the system 100 can adapt to make use of whatever sensor data is available based on which of the one or more sensors 140 are available. For example, if a new computing device is added to the system 100, the new computing device may include additional sensors, which can thereafter be leveraged by the system 100 to further improve generation of an alertness inference.

As described herein, the system 100 generally can be used to generate physiological data associated with a user (e.g., a user of the one or more computing devices 120), such as before or during a sleep session. The physiological data can be analyzed to determine an alertness inference. The one or more sensors 140 can be used to generate, for example, physiological data, audio data, or both. Physiological data generated by one or more of the sensors 140 can be used by the control system 110 to determine a sleep-wake signal associated with the user during the sleep session and an alertness inference. The sleep-wake signal can be indicative of one or more sleep states, including wakefulness, relaxed wakefulness, micro-awakenings, or distinct sleep stages such as, for example, a rapid eye movement (REM) stage, a first non-REM stage (often referred to as “N1”), a second non-REM stage (often referred to as “N2”), a third non-REM stage (often referred to as “N3”), or any combination thereof. Methods for determining sleep states and/or sleep stages from physiological data generated by one or more sensors, such as the one or more sensors 130, are described in, for example, WO 2014/047310, US 2014/0088373, WO 2017/132726, WO 2019/122413, and WO 2019/122414, each of which is hereby incorporated by reference herein in its entirety.

In some implementations, the sleep-wake signal described herein can be timestamped to indicate a time that the user enters the bed, a time that the user exits the bed, a time that the user attempts to fall asleep, etc. The sleep-wake signal can be measured by the one or more sensors 140 during the sleep session at a predetermined sampling rate, such as, for example, one sample per second, one sample per 30 seconds, one sample per minute, etc. In some implementations, the sleep-wake signal can also be indicative of a respiration signal, a respiration rate, an inspiration amplitude, an expiration amplitude, an inspiration-expiration ratio, a number of events per hour, a pattern of events, pressure settings of a respiratory therapy device 112, or any combination thereof during the sleep session. The event(s) can include snoring, apneas, central apneas, obstructive apneas, mixed apneas, hypopneas, a mask leak (e.g., from the user interface), a restless leg, a sleeping disorder, choking, an increased heart rate, labored breathing, an asthma attack, an epileptic episode, a seizure, or any combination thereof. The one or more sleep-related parameters that can be determined for the user during the sleep session based on the sleep-wake signal include, for example, a total time in bed, a total sleep time, a sleep onset latency, a wake-after-sleep-onset parameter, a sleep efficiency, a fragmentation index, or any combination thereof. The physiological data and/or the sleep-related parameters can be analyzed to determine or inform an alertness inference.

The pressure sensor 116 outputs pressure data that can be stored in the memory device 114 and/or analyzed by the one or more processors of the control system 110. In some implementations, the pressure sensor 116 is an air pressure sensor (e.g., barometric pressure sensor) that generates sensor data indicative of the respiration (e.g., inhaling and/or exhaling) of the user and/or ambient pressure. In some implementations, the pressure sensor 116 can be coupled to or integrated in a respiratory therapy device 112 or associated component. The pressure sensor 116 can be, for example, a capacitive sensor, an electromagnetic sensor, a piezoelectric sensor, a strain-gauge sensor, an optical sensor, a potentiometric sensor, or any combination thereof.

The flow rate sensor 118 outputs flow rate data that can be stored in the memory device 114 and/or analyzed by the one or more processors of the control system 110. Examples of flow rate sensors (such as, for example, the flow rate sensor 118) are described in International Publication No. WO 2012/012835, which is hereby incorporated by reference herein in its entirety. In some implementations, the flow rate sensor 118 is used to determine an air flow rate from a respiratory therapy device 112 and/or an air flow rate through a component thereof. In such implementations, the flow rate sensor 118 can be coupled to or integrated in the respiratory therapy device 112 or a component thereof (e.g., the user interface, or the conduit). The flow rate sensor 118 can be a mass flow rate sensor such as, for example, a rotary flow meter (e.g., Hall effect flow meters), a turbine flow meter, an orifice flow meter, an ultrasonic flow meter, a hot wire sensor, a vortex sensor, a membrane sensor, or any combination thereof. In some implementations, the flow rate sensor 118 is configured to measure a vent flow (e.g., intentional “leak”), an unintentional leak (e.g., mouth leak and/or mask leak), a patient flow (e.g., air into and/or out of lungs), or any combination thereof. In some implementations, the flow rate data can be analyzed to determine cardiogenic oscillations of the user.

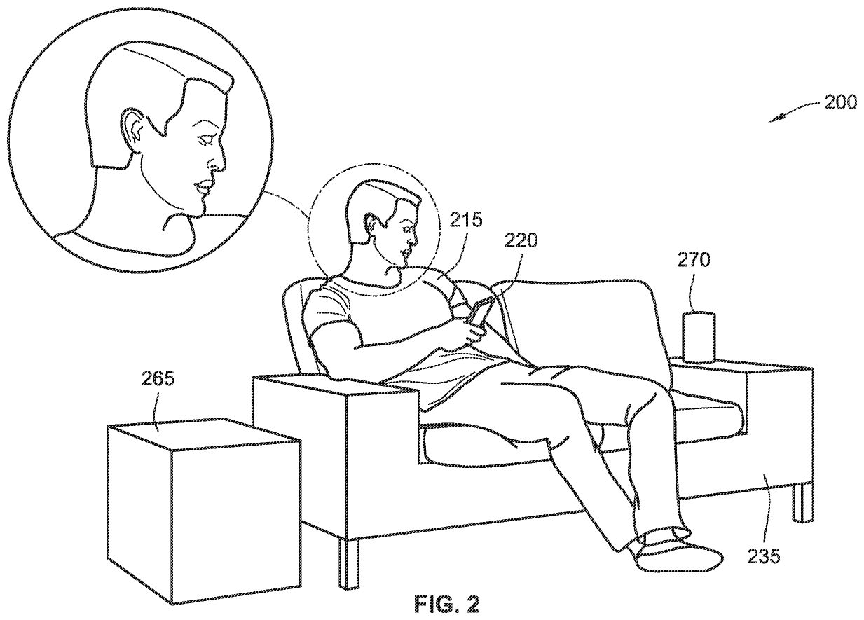

The temperature sensor 142 can generate and/or output temperature data that can be stored in the memory device 114 and/or analyzed by the one or more processors of the control system 110. In some cases, the temperature sensor 142 generates temperatures data indicative of a core body temperature of a user (e.g., a person interacting with at least one of the one or more computing devices 120) of the system 100 (e.g., user 215, 315, 415 of FIGS. 2-4). In some cases, the temperature sensor 142 alternatively or additionally generates temperature data indicative of a skin temperature of the user, an ambient temperature, or any combination thereof. The temperature sensor 142 can be, for example, a thermocouple sensor, a thermistor sensor, a silicon band gap temperature sensor or semiconductor-based sensor, a resistance temperature detector, or any combination thereof or other suitable thermal sensor.

The motion sensor 144 can generate and/or output motion data that can be stored in the memory device 114 and/or analyzed by the one or more processors of the control system 110. The motion sensor 144 is configured to measure motion and/or position of the system 100. In some cases, the motion sensor 144 is an inertial measurement unit (e.g., an inertial measurement chip or the like), an accelerometer, and/or a gyroscope. In some cases, the motion sensor 144 alternatively or additionally generates one or more signals representing bodily movement of the user, from which may be obtained a signal indicative of a level of alertness of the user (e.g., via monitoring hand sway of the user holding the one or more computing devices 120). In some cases, the motion data from the motion sensor 144 can be used in conjunction with additional data from another sensor 140 to generate an alertness inference.

An acoustic sensor 126 can include a microphone 146 and/or a speaker 148. The microphone 146 can generate and/or output sound data that can be stored in the memory device 114 and/or analyzed by the one or more processors of the control system 110. The microphone 146 can be used to record sound(s) (e.g., sounds from the user) to measure (e.g., using the control system 110) one or more biological traits of the user, such as, for example, a respiration signal, a respiration rate, an inspiration amplitude, an expiration amplitude, an inspiration-expiration ratio, a number of events per hour, a pattern of events, a sleep state, a sleep stage, or any combination thereof or other suitable trait. The determined event(s) can include snoring, apneas, central apneas, obstructive apneas, mixed apneas, hypopneas, a restless leg, a sleeping disorder, choking, labored breathing, an asthma attack, an epileptic episode, a seizure, or any combination thereof. Examples of different sleeps states include awake, wakefulness, relaxed wakefulness, drowsy, dozing off (e.g., about to fall asleep), asleep. The sleep state of asleep can include the sleep stage. Examples of different sleep stages include light sleep (e.g., stage N1 and/or stage N2), deep sleep (e.g., stage N3 and/or slow wave sleep), and rapid eye movement (REM) (including, for example, phasic REM sleep, tonic REM sleep, deep to REM sleep transition, and/or light to REM sleep transition). In some cases, sensors other than the microphone 146 can be used instead of or in addition to the microphone 146 to determine an event or sleep state, as described above. In some implementations, the system 100 includes a plurality of microphones (e.g., two or more microphones and/or an array of microphones with beamforming) such that sound data generated by each of the plurality of microphones can be used to discriminate the sound data generated by another of the plurality of microphones.

The speaker 148 can generate and/or output sound waves that are audible to the user. The speaker 148 can be used, for example, as an alarm clock and/or to play an alert or message/notification to the user. In some cases, the microphone 146 and the speaker 148 can be used collectively as a sonar sensor. In such cases, the speaker 148 generates or emits sound waves at a predetermined interval, and the microphone 146 detects the reflections of the emitted sound waves from the speaker 148. The sound waves generated or emitted by the speaker 148 can include frequencies not audible to the human ear, such as infrasonic (e.g., at or below approximately 20 Hz) or ultrasonic (e.g., at or above approximately 18-20 kHz), so as not to disturb the user. Based at least in part on the data from the microphone 146 and the speaker 148, the control system 110 can determine a location of the user and/or biologic traits of the user, such as, for example, a respiration signal, a respiration rate, an inspiration amplitude, an expiration amplitude, an inspiration-expiration ratio, a number of events per hour, a pattern of events, a sleep state, a sleep stage, or any combination thereof or other suitable trait.

The microphone 146 and the speaker 148 can be used as separate devices. In some implementations, the microphone 146 and the speaker 148 can be combined into an acoustic sensor 126 (e.g., a SONAR sensor), as described in, for example, WO 2018/050913 and WO 2020/104465, each of which is hereby incorporated by reference herein in its entirety. In such implementations, the speaker 148 generates or emits sound waves at a predetermined interval and the microphone 146 detects the reflections of the emitted sound waves from the speaker 148. The sound waves generated or emitted by the speaker 148 have a frequency that is not audible to the human ear (e.g., below 20 Hz or above around 18 kHz) so as not to disturb the sleep of the user or a bed partner. Based at least in part on the data from the microphone 146 and/or the speaker 148, the control system 110 can determine a location of the user and/or one or more of the sleep-related parameters described in herein such as, for example, a respiration signal, a respiration rate, an inspiration amplitude, an expiration amplitude, an inspiration-expiration ratio, a number of events per hour, a pattern of events, a sleep state, a sleep stage, pressure settings of the respiratory therapy device 112, or any combination thereof. In such a context, a sonar sensor may be understood to concern an active acoustic sensing, such as by generating and/or transmitting ultrasound and/or low frequency ultrasound sensing signals (e.g., in a frequency range of about 17-23 kHz, 18-22 kHz, or 17-18 kHz, for example), through the air. Such a system may be considered in relation to WO 2018/050913 and WO 2020/104465 mentioned above, each of which is hereby incorporated by reference herein in its entirety. In some cases, the acoustic sensor 126 can be used to identify parameters that are indicative of a particular level of alertness or change in alertness.

In some implementations, the one or more sensors 140 include (i) a first microphone that is the same as, or similar to, the microphone 146, and is integrated in the acoustic sensor 126 and (ii) a second microphone that is the same as, or similar to, the microphone 146, but is separate and distinct from the first microphone that is integrated in the acoustic sensor 126.

The RF sensor 150 includes an RF receiver 152 and/or an RF transmitter 154. The RF transmitter 154 generates and/or emits radio waves having: (i) a predetermined frequency, (ii) a predetermined amplitude (e.g., within a high frequency band, within a low frequency band, long wave signals, short wave signals, etc.), (iii) continuous waves (e.g., continuous wave (CW), frequency modulated CW (FMCW)), (iv) pulsed waves (e.g., pulsed CW, ultrawide band (UWB), and the like), (v) coded waves (e.g., phase-shift keying (PSK), frequency-shift keying (FSK), and the like), or (vi) any combination thereof or other suitable scheme. The RF receiver 152 detects the reflections of the radio waves emitted from the RF transmitter 154, and the detected reflections are output by the RF receiver 152 as data that can be analyzed by the control system 110 to determine a location of the user and/or one or more biological traits, such as, for example, a respiration signal, a respiration rate, an inspiration amplitude, an expiration amplitude, an inspiration-expiration ratio, a number of events per hour, a pattern of events, a sleep state, a sleep stage, or any combination thereof or other suitable trait. The RF receiver 152 and/or the RF transmitter 154 can also be used for wireless communication between the control system 110, the one or more electronic devices 120, the one or more sensors 140, or any combination thereof. While the RF sensor 150 is shown as having a separate RF receiver and RF transmitter in FIG. 1, in some cases, the RF sensor 150 can include a transceiver that acts as both the RF receiver 152 and the RF transmitter 154.

The camera 156 can generate and/or output image data reproducible as one or more images (e.g., still images, video images, or both) that can be stored in the memory device 114 and/or one or more other memory devices. The image data from the camera 156 can be used by the control system 110 to determine one or more biological traits associated with the user interacting with the one or more computing devices 120, such as, for example, head movement (e.g., head sway or bobbing), eye blink rate, eye focus (e.g., a direction of focus or amount of variation in eye focus direction), a heart rate, a blood oxygenation, one or more events (e.g., periodic limb movement or restless leg syndrome), a respiration signal, a respiration rate, an inspiration amplitude, an expiration amplitude, an inspiration-expiration ratio, a number of events per hour, a pattern of events, a sleep state, a sleep stage, or any combination thereof or other suitable trait. In some cases, camera 156 can capture image data in visible light spectrums (e.g., at or approximately 380 nm-740 nm), although that need not always be the case. In some cases, camera 156 can capture infrared light signals or other light signals outside of the visible light range, such as an infrared pattern projected onto the user to facilitate feature recognition of the user's face. However, in some cases, a separate sensor (e.g., infrared sensor 158) can be used for non-visible light ranges (e.g., infrared light).

The infrared (IR) sensor 158 can generate and/or output infrared image data reproducible as one or more infrared images (e.g., still images, video images, or both) or one or more infrared signals, which can be stored in the memory device 114 and/or one or more other memory devices. The infrared data from the IR sensor 158 can be used to determine one or more biological traits, including a temperature of the user and/or movement or motion of the user. The IR sensor 158 can also be used in conjunction with the camera 156 when measuring movement of the user. The IR sensor 158 can detect infrared light having a wavelength between about 700 nm and about 1 mm.

The PPG sensor 160 can generate and/or output physiological data associated with one or more biological traits of the user interacting with the one or more computing devices 120. Such biological traits can include, for example, a heart rate, a heart rate variability, blood oxygenation level, a cardiac cycle, respiration rate, an inspiration amplitude, an expiration amplitude, an inspiration-expiration ratio, a sleep state, a sleep stage, or any combination thereof or other suitable trait. In some cases, the PPG sensor 160 can be worn by the user (e.g., as a wearable watch) and/or embedded in clothing and/or fabric that is worn by the user, although that need not always be the case.

The ECG sensor 128 outputs physiological data associated with electrical activity of the heart of the user. In some implementations, the ECG sensor 128 includes one or more electrodes that are positioned on or around a portion of the user, such as during a sleep session. The physiological data from the ECG sensor 128 can be used, for example, to determine an alertness inference and/or to determine one or more of the sleep-related parameters described herein.

The EEG sensor 130 outputs physiological data associated with electrical activity of the brain of the user. In some implementations, the EEG sensor 130 includes one or more electrodes that are positioned on or around the scalp of the user, such as during a sleep session. The physiological data from the EEG sensor 130 can be used, for example, to determine a sleep state and/or a sleep stage of the user at any given time. In some implementations, the EEG sensor 130 can be integrated in a user interface and/or the associated headgear (e.g., straps, etc.). In some cases, the physiological data from the EEG sensor 130 can be used to determine an alertness inference, such as by identifying levels and/or patterns of electrical activity associated with a given level of alertness or a change in alertness.

The capacitive sensor 162, the force sensor 164, and the strain gauge sensor 166 can generate and/or output data that can be stored in the memory device 114 and used by the control system 110 to determine one or more biological traits, such as those described herein. In some cases, the one or more sensors 140 also include a galvanic skin response (GSR) sensor, an electrocardiogram (ECG) sensor, an electroencephalography (EEG) sensor, an electromyography (EMG) sensor, a blood flow sensor, a respiration sensor, a pulse sensor, a sphygmomanometer sensor, an oximetry sensor, an oxygen sensor, or any combination thereof or other suitable sensor.

The analyte sensor 172 can be used to detect the presence of an analyte in the exhaled breath of the user. The data output by the analyte sensor 172 can be stored in the memory device 114 and used by the control system 110 to determine the identity and concentration of any analytes in the breath of the user. In some implementations, the analyte sensor 172 is positioned near a mouth of the user to detect analytes in breath exhaled from the user's mouth. For example, when a user interface that is a facial mask that covers the nose and mouth of the user is used, the analyte sensor 172 can be positioned within the facial mask to monitor the user's mouth breathing. In other implementations, such as when a user interface that is a nasal mask or a nasal pillow mask is used, the analyte sensor 172 can be positioned near the nose of the user to detect analytes in breath exhaled through the user's nose. In still other implementations, the analyte sensor 172 can be positioned near the user's mouth when a user interface is a nasal mask or a nasal pillow mask. In this implementation, the analyte sensor 172 can be used to detect whether any air is inadvertently leaking from the user's mouth. In some implementations, the analyte sensor 172 is a volatile organic compound (VOC) sensor that can be used to detect carbon-based chemicals or compounds. In some implementations, the analyte sensor 172 can also be used to detect whether the user is breathing through their nose or mouth. For example, if the data output by an analyte sensor 172 positioned near the mouth of the user or within the facial mask (in implementations where a user interface that is a facial mask is used) detects the presence of an analyte, the control system 110 can use this data as an indication that the user is breathing through their mouth. Information from the analyte sensor 172 can be used in the determination of an alertness inference.

The moisture sensor 174 outputs data that can be stored in the memory device 114 and used by the control system 110. The moisture sensor 174 can be used to detect moisture in various areas surrounding the user (e.g., inside or near components of a respiratory therapy device 112, near the user's face, and the like). Thus, in some implementations, the moisture sensor 174 can be coupled to or integrated in a respiratory therapy device 112 or associated component (e.g., user interface or conduit), such as to monitor the humidity of the pressurized air from the respiratory therapy device 112. In other implementations, the moisture sensor 174 is placed near any area where moisture levels need to be monitored. The moisture sensor 174 can also be used to monitor the humidity of the ambient environment surrounding the user, for example, the air inside the bedroom.

The Light Detection and Ranging (LiDAR) sensor 176 can be used for depth sensing. This type of optical sensor (e.g., laser sensor) can be used to detect objects and build three dimensional (3D) maps of the surroundings, such as of a living space. LiDAR can generally utilize a pulsed laser to make time of flight measurements. LiDAR is also referred to as 3D laser scanning. In an example of use of such a sensor, a fixed or mobile device (such as a smartphone) having a LiDAR sensor 176 can measure and map an area extending 5 meters or more away from the sensor. The LiDAR data can be fused with point cloud data estimated by an electromagnetic RADAR sensor, for example. The LiDAR sensor(s) 176 can also use artificial intelligence (AI) to automatically geofence RADAR systems by detecting and classifying features in a space that might cause issues for RADAR systems, such a glass windows (which can be highly reflective to RADAR). LiDAR can also be used to provide an estimate of the height of a person, as well as changes in height when the person sits down, or falls down, for example. LiDAR may be used to form a 3D mesh representation of an environment. In a further use, for solid surfaces through which radio waves pass (e.g., radio-translucent materials), the LiDAR may reflect off such surfaces, thus allowing a classification of different type of obstacles. Data collected from a LiDAR sensor 176 can be used to identify features of an environment, positions of a user in the environment, and/or other features and movements of a user. These features can be used in the determination of an alertness inference, such as by identifying features indicative of a given level of alertness or a change in alertness.

In some implementations, the system 100 also includes a blood pressure (BP) sensor 182. The BP sensor 182 is generally used to aid in generating cardiovascular data for determining one or more blood pressure measurements associated with the user. The BP sensor 182 can include at least one of the one or more sensors 140 to measure, for example, a systolic blood pressure component and/or a diastolic blood pressure component. In some implementations, the BP sensor 182 is a sphygmomanometer including an inflatable cuff that can be worn by the user and a pressure sensor (e.g., the pressure sensor 116 described herein). For example, the BP sensor 182 can be worn on an upper arm of the user. In such implementations where the BP sensor 182 is a sphygmomanometer, the BP sensor 182 also includes a pump (e.g., a manually operated bulb or an electrically operated pump) for inflating the cuff. In some implementations, the BP sensor 182 is coupled to a respiratory therapy device 112, which in turn delivers pressurized air to inflate the cuff. More generally, the BP sensor 182 can be communicatively coupled with, and/or physically integrated in (e.g., within a housing), the control system 110, the memory device 114, and/or one or more computing devices 120.

In other implementations, the BP sensor 182 is an ambulatory blood pressure monitor communicatively coupled to the one of more computing devices 120. An ambulatory blood pressure monitor includes a portable recording device attached to a belt or strap worn by the user and an inflatable cuff attached to the portable recording device and worn around an arm of the user. The ambulatory blood pressure monitor is configured to measure blood pressure between about every fifteen minutes to about thirty minutes over a 24-hour or a 48-hour period. The ambulatory blood pressure monitor may measure heart rate of the user at the same time. These multiple readings are averaged over the 24-hour period. The ambulatory blood pressure monitor determines any changes in the measured blood pressure and heart rate of the user, as well as any distribution and/or trending patterns of the blood pressure and heart rate data during a sleeping period and an awakened period of the user.

In some implementations, the BP sensor 182 is an invasive device which can continuously monitor arterial blood pressure of the user and take an arterial blood sample on demand for analyzing gas of the arterial blood. In some other implementations, the BP sensor 182 is a continuous blood pressure monitor, using a radio frequency sensor and capable of measuring blood pressure of the user 210 once very few seconds (e.g., every 3 seconds, every 5 seconds, every 7 seconds, etc.) The radio frequency sensor may use continuous wave, frequency-modulated continuous wave (FMCW with ramp chirp, triangle, sinewave), other schemes such as PSK, FSK etc., pulsed continuous wave, and/or spread in ultra wideband ranges (which may include spreading, PRN codes or impulse systems).

The measured data and statistics from a BP sensor 182 can be communicated to the one or more computing devices 120 and used in the determination of an alertness inference.

In some implementations, the one or more sensors 140 also include a galvanic skin response (GSR) sensor, a blood flow sensor, a respiration sensor, a pulse sensor, a sphygmomanometer sensor, an oximetry sensor, a sonar sensor, a RADAR sensor, a blood glucose sensor, a color sensor, a pH sensor, an air quality sensor, a tilt sensor, a rain sensor, a soil moisture sensor, a water flow sensor, an alcohol sensor, or any combination thereof.

While shown separately in FIG. 1, one or more of the one or more sensors 140 can be integrated in and/or coupled to any of the other components of the system 100 (e.g., the one or more computing devices 120, the control system 110, the one or more sensors 140, or any combination thereof). For example, the camera 156 and motion sensor 144 can be integrated in and/or coupled to the smartphone 122, the tablet 124, the gaming console 178, or any combination thereof. In some cases, at least one of the one or more sensors 140 are not coupled to the one or more computing devices 120 or the control system 110, and is positioned generally adjacent to the user during use of the one or more computing devices 120. In some cases, the one or more sensors 140 include at least a first sensor in a first computing device (e.g., a smartphone 122) and a second sensor in a second computing device (e.g., a gaming console 178). In such cases, the system 100 can leverage sensor data (e.g., current sensor data or historical sensor data) from one of the first sensor and the second sensor to help generate an alertness inference based on current sensor data of the other of the first sensor and the second sensor.

The display device 190 of the system 100 can generally be used to display image(s) including still images, video images, projected images, holograms, interactive images, or the like, or any combination thereof; and/or information regarding the one or more computing devices 120. For example, the display device 190 can provide information regarding the status of the one or more computing devices 120 and/or other information (e.g., a message). In some cases, the display device 190 is included in and/or is a portion of the computing device 120 (e.g., a touchscreen of a smartphone 122 or tablet 134, or a screen coupled to or housed in a gaming console 178).

The input device 192 of the system 100 can be generally used to receive user input to enable user interaction with the control system 110, the memory 114, the one or more computing devices 120, the one or more sensors 140, or any combination thereof. The input device 192 can include a microphone for speech (e.g., the microphone 146), a touch-sensitive screen for gesture or graphical input, a keyboard, a mouse, a motion input (e.g., the motion sensor 144, the camera 156), or any combination thereof or other suitable input. In some cases, the input device 192 includes multimodal systems that enable a user to provide multiple types of input to communicate with the system 100. The input device 192 can alternatively or additionally include a button, a switch, a dial, or the like to allow the user to interact with the system 100. The button, the switch, the dial, or a similar element may be a physical structure or a virtual structure (e.g., software application accessible via an input such as a touch-sensitive screen or motion input). In some cases, the input device 192 may be arranged to allow the user to select a value and/or a menu option. In some cases, the input device 192 is included in and/or is a portion of the computing device 120 (e.g., a touchscreen of a smartphone 122 or tablet 134, or a controller or embedded button set of a gaming console 178).

In some cases, the input device 192 includes a processor, a memory, and a display device, that are the same as, or similar to, the processor(s) of the control system 110, the memory device 114, and the display device 190. In some cases, the processor and the memory of the input device 192 can be used to perform any of the respective functions described herein for the processor and/or the memory device 114. In some cases, the control system 110 and/or the memory 114 is integrated in the input device 192.

The display device 190 alternatively or additionally acts as a human-machine interface (HMI) that includes a graphic user interface (GUI) configured to display the image(s) and an input interface. The display device 190 can be an LED display, an OLED display, an LCD display, or the like. The input interface can be, for example, a touchscreen or touch-sensitive substrate, a mouse, a keyboard, or any sensor system configured to sense inputs made by a human user interacting with the system 100 with or without direct user contact/touch.

While the display device 190 and the input device 192 is described and depicted in FIG. 1 as being separate and distinct components of the system 100, in some implementations, the display device 190 and/or the input device 192 are integrated in and/or directly coupled to one or more of the one or more computing devices 120 and/or one or more of the one or more sensors 140, and/or the control system 110, and/or the memory 114.

While the system 100 is shown as including all of the components described herein with respect to FIG. 1, more or fewer components can be included in a system for generating and leveraging an alertness inference based on received (e.g., collected or otherwise received) interaction data. For example, a system 100 may include only a smartphone 122 incorporating a control system 110, a memory 114, a display device 190, an input device 192, a microphone 146, a speaker 148, a camera 156, and a motion sensor 144. Other arrangements of components can be included in a suitable system. Thus, various systems for generating and leveraging an alertness inference associated with a user interacting with one or more computing devices can be formed using any portion or portions of the components shown and described herein and/or in combination with one or more other components.

FIG. 2 is a perspective view of a user 215 interacting with a computing device with a high level of alertness, according to certain aspects of the present disclosure. The computing device can be any suitable computing device, such as any of the one or more computing devices 120 of FIG. 1, although as depicted in FIG. 2, the computing device is a smartphone 220. The user 215 can be located in an environment 200, such as a room, region of a room, a building, a facility, or other environment. The user 215 can be standing, sitting, reclining, lying, or otherwise positioned in the environment 200. As depicted in FIG. 2, the user is reclining on a sofa 235. The environment 200 can include any suitable objects, such as furniture (e.g., side table 265), other computing devices (e.g., smart speaker 270), light sources (e.g., exterior light sources, such as sunlight or moonlight, or interior light sources, such as light bulbs), and other individuals.

Under normal use (e.g., the current settings of the smartphone 220 without taking into account an alertness inference), the smartphone 220 can receive messages and present messages in a particular fashion, such as using a particular presentation scheme (e.g., present messages with audio and visual indicators).

While the user 215 is interacting with the smartphone 220, the smartphone 220 receives interaction data. The interaction data may be received from sensors within the smartphone 220 (e.g., a camera and a motion sensor), from software within the smartphone 220 (e.g., data associated with user inputs and reaction times), or from a remote source (e.g., a microphone in a smart speaker 270 or other source). The smartphone 220 can use the interaction data to generate an alertness inference.

Since user 215 has a high level of alertness, the interaction data received by the smartphone 220 in FIG. 2 can be indicative of this high level of alertness. The interaction data can include biometric data, inertia data, and/or software-usage data. For example, the user 215 may exhibit a particular blink rate (e.g., a normal blink rate), eye focus (e.g., steady eye focus on the screen of the smartphone 220), breathing rate (e.g., a normal breathing rate), and head sway (e.g., low head sway or bobbing) that can be collected as biometric data and can be indicative of a high level of alertness. In another example, the smartphone 220 may detect particular motion in space of the smartphone 220 (e.g., only small, subtle motion indicative of a user holding the smartphone 220 steadily in a hand), a particular orientation of the smartphone 220 (e.g., in an orientation where the display faces the face of the user 215), and other such inertial information that can be collected as inertia data and can be indicative of a high level of alertness of a user interacting with the smartphone 220. In another example, the smartphone 220 may detect particular reaction speeds (e.g., rapid reaction after a prompt appears onscreen), button press accuracies (e.g., accurate button presses with low variance), and app usage (e.g., use of a app that inherently requires a certain amount of attention) that can be collected as software-usage data and can be indicative of a high level of alertness. Other examples and combinations of data can be used as biometric data, inertia data, and/or software-usage data.

Based on the received interaction data, the smartphone 220 can generate an alertness inference of user 215 interacting with the smartphone 220. The alertness inference can be indicative of a high level of alertness, and can be stored and/or presented as a high alertness score (e.g., a numerical score, such as an 80 out of 100, although any scale can be used), a high alertness categorization (e.g., “fully alert”), or other suitable methodology. Based on this alertness inference indicating a high level of alertness, the smartphone 220 can permit received messages to be presented as normal.

In some cases, the alertness inference can be stored with historical information associated with the user's interactions with the smartphone 220. For example, the user's high level of alertness can be indicative that the actions being taken on the phone (e.g., the app being used or the type of action being undertaken, such as typing or email drafting) are of a high level of importance. Thus, an importance score for a particular action can be generated using the alertness inference. This importance score can be used to control presentation of messages on the smartphone 220.

In some cases, the alertness inference can also be used to generate a receptiveness score. The user's high level of alertness while using the smartphone 220 can be indicative that the user will or will not be receptive to a new message or particular type of new message, such as particular advertising content. Based on the receptiveness score, the smartphone 220 can choose whether or not to present certain messages, such as advertising content. In some cases, the smartphone 220 can select a particular message for display from a set of messages based on the alertness inference, an importance score, and/or a receptiveness score.

In some cases, the computing device (e.g., smartphone 220) can receive data about the environment 200, which can be further used to generate or confirm an alertness inference or other score. For example, a high level of light in an environment 200 can be suggestive that the user 215 may have a high level of alertness.

While the user 215 has a high level of alertness in FIG. 2, that level of alertness may change over time. The smartphone 220 can track such changes over time, as described herein.

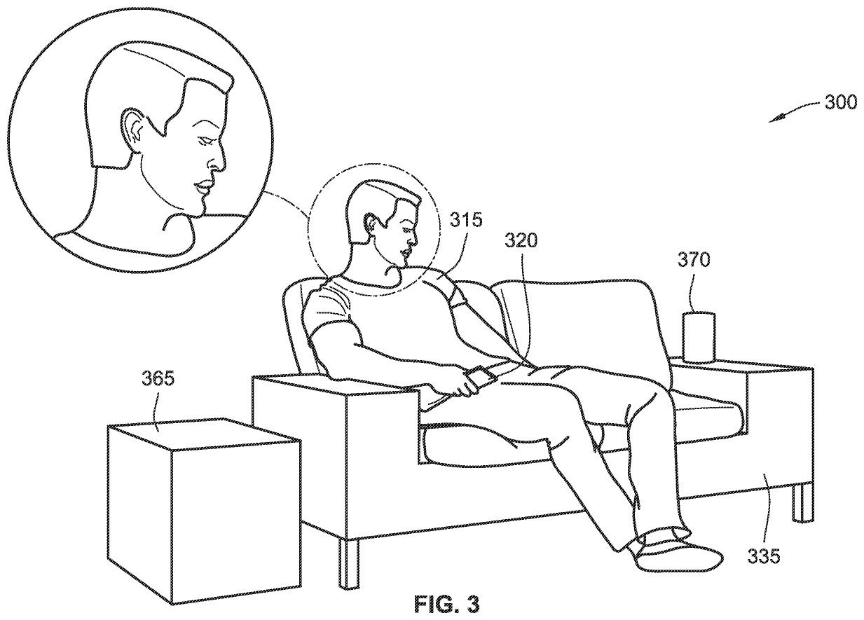

FIG. 3 is a perspective view of a user 315 interacting with a computing device with a low level of alertness, according to certain aspects of the present disclosure. The user 315 can be user 215 of FIG. 2 after a change in alertness. Likewise, the environment 300 and other elements of the environment 300 (e.g., smartphone 320, smart speaker 370, side table 365, and sofa 335) may be the same as environment 200 and the other elements of environment 200 (e.g., smartphone 220, smart speaker 270, side table 265, and sofa 235), respectively.

The low level of alertness of user 315 can be lower than the high level of alertness of user 215 of FIG. 2. As depicted in FIG. 3, the user is reclining on a sofa 335 (e.g., reclining further than user 215 of FIG. 2). Due to the user's lower level of alertness, the user 315 may be interacting with the smartphone 320 in a different fashion than when at a high level of alertness. For example, the user 315 may not hold the smartphone 320 as securely and/or steadily, and may rely on other objects for support (e.g., the user's leg), the user 315 may not hold eye contact and/or focus with the screen of the smartphone 320, and the user 315 may not respond to prompts on the smartphone 320 as quickly.

While the user 315 is interacting with the smartphone 320, the smartphone 320 receives interaction data. The interaction data may be received from sensors within the smartphone 320 (e.g., a camera and a motion sensor), from software within the smartphone 320 (e.g., data associated with user inputs and reaction times), or from a remote source (e.g., a microphone in a smart speaker 370 or other source). The smartphone 320 can use the interaction data to generate an alertness inference.

Since user 315 has a low level of alertness, the interaction data received by the smartphone 320 in FIG. 3 can be indicative of this low level of alertness. The interaction data can include biometric data, inertia data, and/or software-usage data. For example, the user 315 may exhibit a particular blink rate (e.g., a higher-than-usual blink rate), eye focus (e.g., unsteady eye focus on the screen of the smartphone 320), breathing rate (e.g., a slower-than-normal breathing rate), and head sway (e.g., significant head sway or bobbing) that can be collected as biometric data and can be indicative of a low level of alertness. In another example, the smartphone 320 may detect particular motion in space of the smartphone 320 (e.g., larger motion indicative of a user holding the smartphone 320 unsteadily in a hand or letting the smartphone 320 repeatedly droop towards the floor), a particular orientation of the smartphone 320 (e.g., in an orientation where the display does not directly face the face of the user 315), and other such inertial information that can be collected as inertia data and can be indicative of a low level of alertness of a user interacting with the smartphone 320. In another example, the smartphone 320 may detect particular reaction speeds (e.g., slow reaction after a prompt appears onscreen), button press accuracies (e.g., inaccurate button presses with high variance), and app usage (e.g., use of a app that does not inherently require any significant amount of attention) that can be collected as software-usage data and can be indicative of a low level of alertness. Other examples and combinations of data can be used as biometric data, inertia data, and/or software-usage data.

Based on the received interaction data, the smartphone 320 can generate an alertness inference of user 315 interacting with the smartphone 320. The alertness inference can be indicative of a low level of alertness, and can be stored and/or presented as a low alertness score (e.g., a numerical score, such as a 30 out of 100, although any scale can be used), a low alertness categorization (e.g., “dozing off”), or other suitable methodology. Based on this alertness inference indicating a low level of alertness, the smartphone 320 can stop received messages from being presented as normal (e.g. according to default settings or rules), instead altering the presentation of the messages to either withhold the message or present the message using a different presentation scheme.

In some cases, the alertness inference can be stored with historical information associated with the user's interactions with the smartphone 320. For example, the user's low level of alertness can be indicative that the actions being taken on the phone (e.g., the app being used or the type of action being undertaken, such as watching a movie or playing a game) are of a low level of importance. Thus, an importance score for a particular action can be generated using the alertness inference. This importance score can be used to control presentation of messages on the smartphone 320.

In some cases, the alertness inference can also be used to generate a receptiveness score. The user's low level of alertness while using the smartphone 320 can be indicative that the user will or will not be receptive to a new message or particular type of new message, such as particular advertising content. Based on the receptiveness score, the smartphone 320 can choose whether or not to present certain messages, such as advertising content. In some cases, the smartphone 320 can select a particular message for display from a set of messages based on the alertness inference, an importance score, and/or a receptiveness score. In some cases, detection of hypnagogic jerk (e.g., sleep start—a form of involuntary muscle twitch known as myoclonus) may be indicative of low receptiveness.

In some cases, the computing device (e.g., smartphone 320) can receive data about the environment 300, which can be further used to generate or confirm an alertness inference or other score. For example, a low level of light in an environment 300 can be suggestive that the user 315 may have a low level of alertness.

While under normal use (e.g., the current settings of the smartphone 320 without taking into account an alertness inference), the smartphone 320 may receive messages and present messages in a particular fashion, such as using a particular presentation scheme (e.g., present messages with audio and visual indicators), because the user 315 is exhibiting a low level of alertness, the smartphone 320 may alter presentation of the messages.

In some cases, the alertness inference can be used to alter presentation of a message by determining a particular presentation scheme to use when presenting the message. While a normal presentation scheme may involve presenting the message using audio and visual indicators (e.g., a drop-down display and an audible tone), alerting presentation of the message can include presenting the message using an alternate presentation scheme, which may use any combination of outputs designed to present the message to the user 315 (e.g., present the content of the message or make the user 315 aware of the existence of the message). For example, an alternate presentation scheme can include only visually presenting a message on the smartphone 320, without any audio indicator. In some cases, an alternate presentation scheme can involve presenting a message using an alternate route of presentation. For example, while the message may normally be presented using the display and speaker of the smartphone 320, altering presentation of the message can involve selecting an alternate route of presentation, such as presenting the message using another computing device, such as a smart speaker 370. In such an example, instead of presenting the message on the smartphone 320, the message may be presented by generating a tone or outputting spoken text (e.g., computer-generated speech) associated with the message using the smart speaker 370. An alternate route of presentation can be especially useful when a computing device determines that presentation through the computing device would not be especially successful due to the low level of alertness exhibited by the user interacting with the computing device.

In some cases, an alertness inference generated by the computing device can be indicative of an overall alertness level of the user. For example, as seen in FIG. 3, user 315 is shown dozing off, and therefore the alertness inference of a low level of alertness, as determined by smartphone 320, is indicative of the user's overall alertness level. However, that need not always be the case. In some cases, the alertness inference generated by the computing device is indicative of an alertness level associated with the user interacting with the computing device. For example, if a user were to be interacting with a smartphone but mostly paying attention to another device (e.g., television) or individual (e.g., a spouse), the smartphone may generate an alertness inference that the user has a low level of alertness associated with the user's interaction with the smartphone, but that low level of alertness may not necessarily correlate to the user's overall level of alertness, which may still be high since the user is interacting with other devices or individuals. In some cases, generating an alertness inference can include using the interaction data to infer whether or not the user is interacting with another object (e.g., another device or an individual). For example, if the computing device determines that the user is likely not interacting with another object, a determination can be made that the alertness inference correlates with the user's overall alertness level.

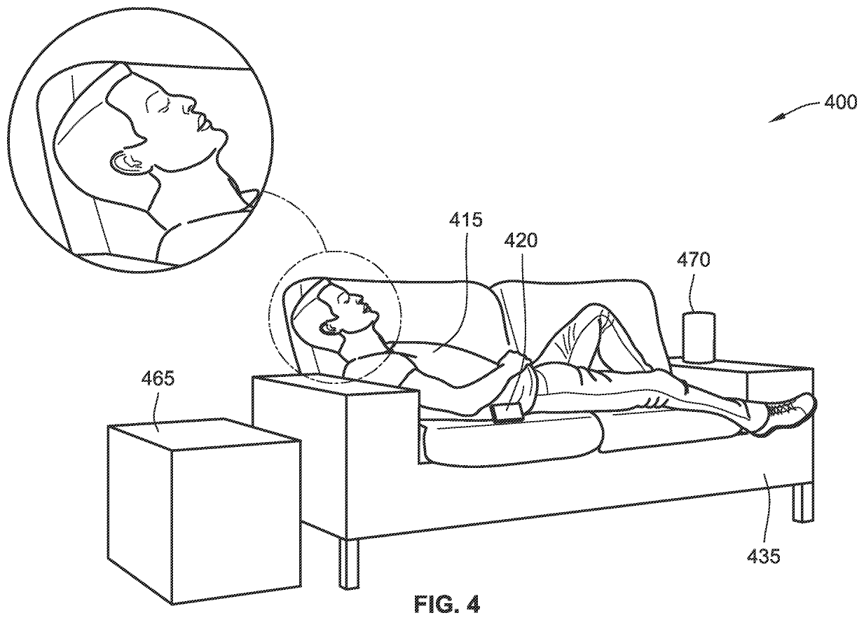

FIG. 4 is a perspective view of a user 415 that has fallen asleep after interacting with a computing device, according to certain aspects of the present disclosure. The user 415 can be user 215 or user 315 of FIG. 2 or 3, respectively, after a change in alertness. Likewise, the environment 400 and other elements of the environment 400 (e.g., smartphone 420, smart speaker 470, side table 465, and sofa 435) may be the same as environment 200 and the other elements of environment 200 (e.g., smartphone 220, smart speaker 270, side table 265, and sofa 235), respectively.

Since user 415 has fallen asleep, the level of alertness of user 415 can be determined to be extremely low. This extremely low level of alertness of user 415 can be lower than the high level of alertness of user 215 of FIG. 2 and lower than the low level of alertness of user 315 of FIG. 3. As depicted in FIG. 4, the user is lying down on a sofa 435 (e.g., reclining even further than user 315 of FIG. 3). Due to the user's extremely low level of alertness, the user 415 may no longer be actively interacting with the smartphone 420. For example, the user 415 may no longer be holding the smartphone 420, and may allow the smartphone 420 to fall and/or rest on other objects (e.g., the user's body, the sofa 435, or the floor), the user 415 may make no eye contact and/or focus with the screen of the smartphone 420, and the user 415 may not respond to prompts on the smartphone 420.

While the user 415 is passively interacting with the smartphone 420 (e.g., for a duration after the user has stopped actively interacting with the smartphone 420), the smartphone 420 receives interaction data. Due to the user 415 ceasing active interaction with the smartphone 410, the interaction data may be indicative of a lack of active interaction. As used herein, the terms “interaction” and “interaction data” are inclusive of a lack of active interaction for at least a period following and/or preceding a user actively interacting with the computing device. The interaction data may be received from sensors within the smartphone 420 (e.g., a camera and a motion sensor), from software within the smartphone 420 (e.g., data associated with user inputs and reaction times), or from a remote source (e.g., a microphone in a smart speaker 470 or other source). The smartphone 420 can use the interaction data to generate an alertness inference.

Since user 415 has an extremely low level of alertness, the interaction data received by the smartphone 420 in FIG. 4 can be indicative of this extremely low level of alertness. The interaction data can include biometric data, inertia data, and/or software-usage data. For example, the sensors may detect no blink rate, no eye focus, no breathing rate or a particular breathing rate (e.g., slow breathing consistent with sleep), no head sway that can be collected as biometric data and can be indicative of an extremely low level of alertness. In another example, the smartphone 420 may detect particular motion in space of the smartphone 420 (e.g., a falling motion followed by steadiness indicative of resting on a surface such as the sofa 435 or floor, or a rhythmic motion indicative of resting on the body of a breathing user 415), a particular orientation of the smartphone 420 (e.g., in an orientation where the display is face down or face up, such as if resting on the sofa 435 or floor, or otherwise not directly facing the face of the user 415), and other such inertial information that can be collected as inertia data and can be indicative of an extremely low level of alertness of a user interacting with the smartphone 420. In another example, the smartphone 420 may detect a lack of reactions (e.g., no reaction after a prompt appears onscreen), a lack of button presses, and app usage (e.g., use of a app that is designed to induce and/or monitor sleep) that can be collected as software-usage data and can be indicative of an extremely low level of alertness. Other examples and combinations of data can be used as biometric data, inertia data, and/or software-usage data.

Based on the received interaction data, the smartphone 420 can generate an alertness inference of user 415 interacting with the smartphone 420. The alertness inference can be indicative of an extremely low level of alertness, and can be stored and/or presented as an extremely low alertness score (e.g., a numerical score, such as a 5 out of 100, although any scale can be used), a low alertness categorization (e.g., “asleep”), or other suitable methodology. Based on this alertness inference indicating an extremely low level of alertness, the smartphone 420 can stop received messages from being presented as normal, instead altering the presentation of the messages to either withhold the message or present the message using a different presentation scheme. For example, messages can be withheld until a subsequent alertness inference is generated indicating that the user 415 has awaken or at least achieved a level of alertness greater than a certain threshold.

In some cases, the alertness inference can be used to store historical information associated with the user's interactions with the smartphone 420. For example, the user's extremely low level of alertness can be indicative that the actions being taken on the phone (e.g., the app being used or the type of action being undertaken just prior to this extremely low level of alertness, such as watching a movie or playing a game) are of a low level of importance. Thus, an importance score for a particular action can be generated using the alertness inference. This importance score can be used to control presentation of messages on the smartphone 420.

In some cases, the alertness inference can also be used to generate a receptiveness score. The user's extremely low level of alertness while using the smartphone 420 can be indicative that the user will not be receptive to a new message or particular type of new message, such as particular advertising content. In some cases, the receptiveness score associated with a particular type or piece of advertising content can be based on interaction data that includes a type of app being used, the app being used, a piece of content being viewed (e.g., a movie, book, or webpage), or any combination thereof. Based on the receptiveness score, the smartphone 420 can choose to not present certain messages, such as advertising content. In some cases, the smartphone 420 can select a particular message for display from a set of messages based on the alertness inference, an importance score, and/or a receptiveness score. For example, a particular message, such as one appropriate for an individual who is waking up, can be selected for display based on an alertness inference indicating an extremely low level of alertness followed by a subsequent alertness inference indicating a higher level of alertness indicative of the user awaking and interacting with the computing device.

In some cases, the computing device (e.g., smartphone 420) can receive data about the environment 400, which can be further used to generate or confirm an alertness inference or other score. For example, an extremely low level of light in an environment 400 can be suggestive that the user 415 may have an extremely low level of alertness.

While under normal use (e.g., the current settings of the smartphone 420 without taking into account an alertness inference), the smartphone 420 may receive messages and present messages in a particular fashion, such as using a particular presentation scheme (e.g., present messages with audio and visual indicators), because the user 415 is exhibiting an extremely low level of alertness, the smartphone 420 may alter presentation of the messages. For example, messages with a high level of importance (e.g., a high importance score or otherwise essential messages) can be presented in a fashion designed to attract the attention of the sleeping user 415, such as through a loud, audible alert played from the smartphone 420 or smart speaker 470.

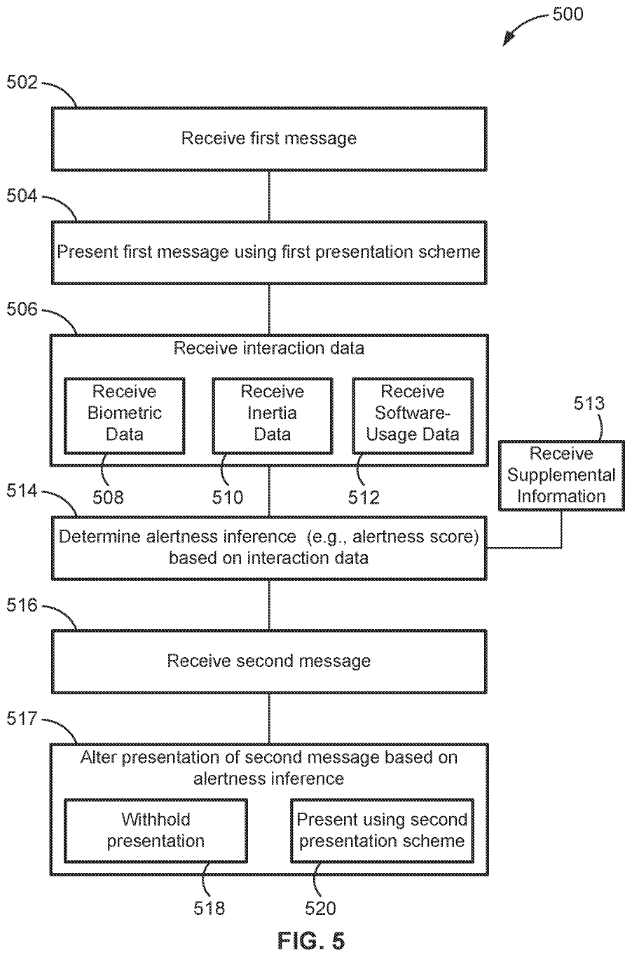

FIG. 5 is a flowchart depicting a process 500 for monitoring and leveraging alertness, according to certain aspects of the present disclosure. Process 500 can be performed using system 100 of FIG. 1, such as on a computing device (e.g., the one or more computing devices 120 of FIG. 1). At block 502, a first message is received. The message can be from any suitable source, such as software operating on the computing device, software associated with other elements of the system, software operating on a remote device (e.g., a server or cloud-based computing device), or an individual (e.g., a text message sent from a computing device of another individual). At block 504, the message can be presented using a first presentation scheme. Presentation of the message using the first presentation scheme can include presenting the message would normally occur without taking into account any interaction inference. For example, a computing device can include rules (e.g., notification settings) that define how incoming messages are presented. In some cases, such rules can include non-dynamic rules for presenting messages, such as do-not-disturb settings not based on interaction data. Blocks 502 and 504 are optional parts of process 500 and are used to illustrate how presentation of a second message can be altered based on an alertness inference. In some cases, for example, a previous alertness inference may have already been generated prior receiving the first message at block 502, in which case presentation of the first message using the first presentation scheme at block 504 may occur as a result of the computing device determining that no alteration to the presentation of the message is warranted given the previous alertness inference.

At block 506, interaction data can be received. Receiving interaction data can include collecting and/or sensing interaction data, such as through sensors (e.g., one or more sensors 140 of FIG. 1), receiving interaction data from software operating on the computing device, and/or receiving interaction data via a communication link, such as a network connection (e.g., a local area network, a personal area network, or the like). In some cases, receiving interaction data can include receiving interaction data from a wearable device (e.g., a smart watch or other wearable sensor).

Receiving interaction data at block 506 can include receiving biometric data at block 508, receiving inertia data at block 510, receiving software-usage data at block 512, or any combination thereof. In some cases, receiving interaction data can include pre-processing sensor data to obtain the biometric data, inertia data, and/or software-usage data. For example, sensor data associated with capture of light reflected form a user's face (e.g., via a camera) may be pre-processed or otherwise analyzed to identify the frequency of eye blinks (e.g., spontaneous eye blink rate) of the user. The biometric data received at block 508, inertia data received at block 510, and software-usage data received at block 512 can be biometric data, inertia data, and software-usage data as disclosed herein, respectively.

At block 514, an alertness inference can be determined. Determining an alertness inference can include generating the alertness inference using the interaction data. In some cases, the alertness inference can be generated by using a portion of the interaction data, in which case the alertness inference can be confirmed or refuted using a second portion of the interaction data (e.g., a remainder or part of a remainder of the interaction data). For example, in some cases, software-usage data received at block 512 can be used to confirm or refute an alertness inference generated using biometric data and/or inertia data received at blocks 508 and 510, respectively.

Generating the alertness inference can include applying one or more weighted formulae to the received interaction data to generate the alertness inference. In some cases, generating the alertness inference can include applying the inference data to an algorithm, such as a machine learning algorithm or deep neural network, to generate the alertness inference.

The alertness inference generated at block 514 can take any suitable form. In some cases, the alertness inference can be generated, stored, and/or output as a number, such as an alertness score. An alertness score can include a range extending from fully not alert (e.g., deeply asleep) to fully alert (e.g., hyper-alert). Any suitable scale can be used, such as a numerical scale from 0 to 100, with higher numbers indicating a greater level of alertness. Other scoring techniques can be used. In some cases, an alertness inference can include an alertness categorization, which can include lumping adjacent levels of alertness into the same overall alertness category. For example, an alertness inference including an alertness categorization can categorize the levels of alertness into various enumerated levels as described herein (e.g., “asleep,” “dozing,” and “fully awake”).

At optional block 513, supplemental information can be received. Receiving supplemental information can include collecting and/or sensing supplemental information, such as through sensors (e.g., one or more sensors 140 of FIG. 1), receiving supplemental information from software operating on the computing device, and/or receiving supplemental information via a communication link, such as a network connection (e.g., a local area network, a personal area network, or the like). In some cases, receiving supplemental information can include receiving supplemental information from a wearable device (e.g., a smart watch or other wearable sensor).

Supplemental can include additional information accessible to the computing device. Supplemental information may not be associated with the user's interaction with the computing device, although that need not always be the case. Examples of supplemental information can include a current time (e.g., time of day), a geolocation (e.g., a location within a frame of reference, such as a location on the earth, a location in a facility, or a location in a house), a current time zone, power data from the computing device (e.g., a power level, a power saving mode, an app mode, a device mode, a CPU status, whether or not the device is being charged, an estimated amount of usable battery life remaining), an ambient light level, or any other suitable information. In some cases, supplemental information can include information related to a user travelling from one location to another location, such as via a vehicle (e.g., a bus or train). For example, supplemental information can include calendar data, ticket data, itinerary data, or the like.

In some cases, determining the alertness inference at block 514 can be based on the supplemental information received at block 513. In some cases, the supplemental information can be used to help generate, confirm, and/or refute an alertness inference.

At block 516, a second message is received. The message can be from any suitable source, such as software operating on the computing device, software associated with other elements of the system, software operating on a remote device (e.g., a server or cloud-based computing device), or an individual (e.g., a text message sent from a computing device of another individual). Under normal conditions not taking into account the alertness inference, the second message may otherwise have been presented, such as using the first presentation scheme of block 504. However, at block 517, presentation of the second message is altered based on the alertness inference.

Altering presentation of the second message at block 517 can include withholding presentation of the message at block 518 or presenting the message using a second presentation scheme at block 520. Withholding the message at block 518 can include withholding the message as disclosed herein, optionally further including re-delivering (e.g., re-attempting delivery of) the message at a later time. The presenting using a second presentation scheme at block 520 can include presenting the message in a different fashion than if the message were presented using the first presentation scheme. Any differences in presentation can be used. For example, where the first presentation scheme involves generating visual and audio indications of the message (e.g., a notification with visual and audio components), the second presentation scheme may involve generating only a visual indication of the message without an audio indication, or generating a background indication of the message (e.g., setting a flag on a home screen for the user to view at a later time or delivering a notification to a notification tray without an on-screen notification).

In some cases, altering presentation of the second message block at 517 can include temporarily altering a rule (e.g., a notification setting) of the computing device. In some cases, temporarily altering the rule can occur for only the second message, for all incoming messages of a certain type (e.g., app alerts or text message), for all incoming messages from a particular source (e.g., from a particular app or a particular individual), or for all incoming messages.

While depicted in a particular top-down order in FIG. 5, the various blocks of process 500 can be performed in any suitable order. For example, in some cases, upon determining the alertness inference, the presentation of a second message can be altered before the second message is received. In other words, determining of the alertness inference can automatically adjust one or more rules for presenting future messages. For example, the one or more rules can be adjusted until a subsequent alertness inference is made that is sufficiently different from the current alertness inference (e.g., a user awakens after being asleep for a period of time).

In some cases, altering presentation of a message can include applying an existing rule (e.g., notification setting) that makes use of an alertness inference. For example, instead of adjusting an existing rule, altering presentation of the message at block 517 can include applying the alertness inference determined at block 514 to a rule, in which case that rule would change how a message is presented depending on the alertness inference. For example, a rule can be set to present messages when the alertness inference shows the user has a high level of alertness, but can be set to withhold messages when the alertness inference shows the user has a low level of alertness.

Process 500 of FIG. 5 is described as including receiving of a first message (e.g., at block 502) and receiving of a second message (e.g., at block 516) for illustrative purposes to help describe certain aspects of the disclosure. While the first message can be received shortly before the second message, that need not always be the case, and the first message may be received minutes, hours, days, weeks, months, or years before the second message. Further, the first message may be presented at block 504 using the first presentation scheme either because no alertness inference had yet been made, or because an alertness inference had been previously made and a determination was made to present the first message accordingly. In some cases, presentation of (and responsiveness to) the first message can be used to affect presentation of the second message, as described herein.

In some cases, process 500 can begin at block 506 without a first message having been previously received. In such examples, the interaction data received at block 506 can be used to determine an alertness inference, which is then used to control how a received message (e.g., “second” message received at block 516, which may in this case be the first message received) is presented. For example, physiological data, such as received biometric data, can be used to determine an alertness inference of a user, even before a message is presented to a user. The alertness inference can then be used to control how to present an initial message, and optionally subsequent messages, to the user.

Additionally, process 500 describes presentation of the second message as being altered based on the alertness inference for illustrative purposes to help describe certain aspects of the disclosure. In use, sometimes presentation of a second message may be not altered (e.g., presented using the first presentation scheme) if the alertness inference is such that no alteration is warranted.

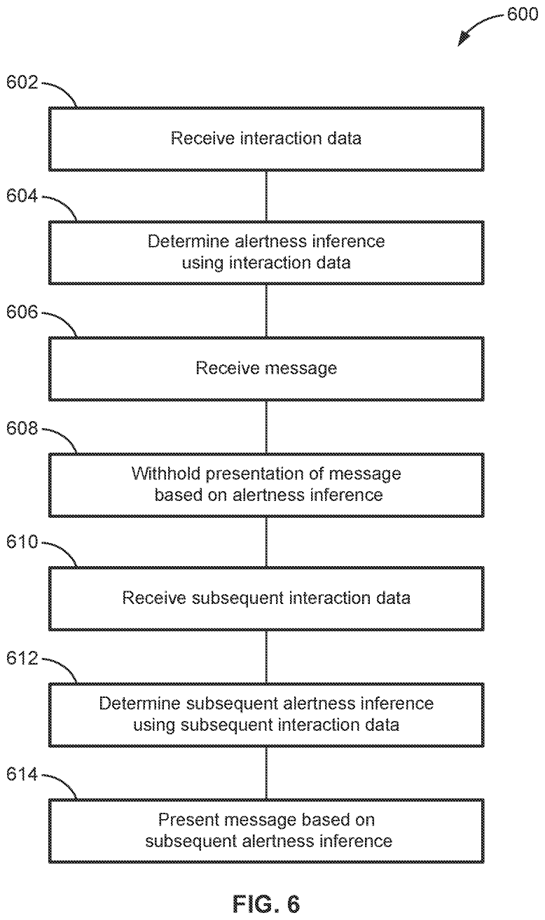

FIG. 6 is a flowchart depicting a process 600 for controlling presentation of a message based on monitored alertness, according to certain aspects of the present disclosure. Process 600 can be performed by system 100 of FIG. 1, such as by a computing device (e.g., the one or more computing devices 120 of FIG. 1).

At block 602, interaction data can be received. Receiving interaction data at block 602 can be related to any suitable interaction between the user and the computing device, such as interactions associated with a message (e.g., first message or second message of process 500 of FIG. 5), interactions associated with a particular app on the computing device, or the like. At block 604, an alertness inference can be determined. Receiving interaction data at block 602 and determining an alertness inference at block 604 can be similar to receiving interaction data at block 506 and determining an alertness inference at block 514 of FIG. 5, respectively. At block 606, a message can be received. Receiving a message at block 606 can be similar to receiving the second message at block 516 of FIG. 5. At block 608, presentation of the message can be withheld based on the alertness inference. Withholding presentation of the message at block 608 can be similar to withholding presentation of the message at block 518 of FIG. 5. In some cases, instead of withholding presentation of the message at block 608, the message can be presented using an alternate presentation scheme. In such cases, the alternate presentation scheme can a presentation scheme for which a user may nonetheless want a supplemental presentation of the message again, as disclosed herein. For example, a user may set up the system to present messages only visually while the user has an alertness score below a threshold (e.g., drowsy or asleep), but present those same messages again, optionally with a different presentation scheme, after the system has determined that the user's alertness score has changed sufficiently (e.g., the user awoke and is sufficiently alert).

At block 610, subsequent interaction data can be received. Receiving subsequent interaction data at block 610 can be similar to receiving interaction data at block 602, however at a later point in time. In some cases, subsequent interaction data can optionally include historical interaction data, which can include the interaction data previously received at block 602.

At block 612, a subsequent alertness inference can be determined using the subsequent interaction data from block 610. Determining the subsequent alertness inference at block 612 can be similar to determining the alertness inference at block 604, however using the subsequent interaction data. The subsequent alertness inference can be different than the previous alertness inference, such as indicative that the user is more alert than before. At block 614, the message (e.g., the previously withheld message) can be presented based on the subsequent alertness inference.

In an example especially suitable for process 600, a text message may be sent to the smartphone of a user who has fallen asleep, in which case the smartphone would withhold presenting a notification of the text message arrival until after the smartphone has determined that the user is awake and sufficiently alert. In such an example, the alertness inference determined at block 604 can be indicative that the user has a relatively low level of alertness (e.g., the user is drowsy or possibly sleeping), and therefore a decision can be made to not present the received message (e.g., by withholding presentation of the message at block 608). However, at a later point in time, the subsequent alertness inference can be indicative that the user has a higher, sufficiently high, or relatively high level of alertness (e.g., the user is awake and interacting with the phone), at which point the system can decide to present the previously withheld message (e.g., by presenting the message at block 614).

The subsequent interaction data and subsequent alertness inference from blocks 610 and 612 can be immediately subsequent the interaction data and alertness inference from blocks 602 and 604, although that need not be the case. In some cases, any number of actions can occur between blocks 602 and 604 and blocks 610 and 612.

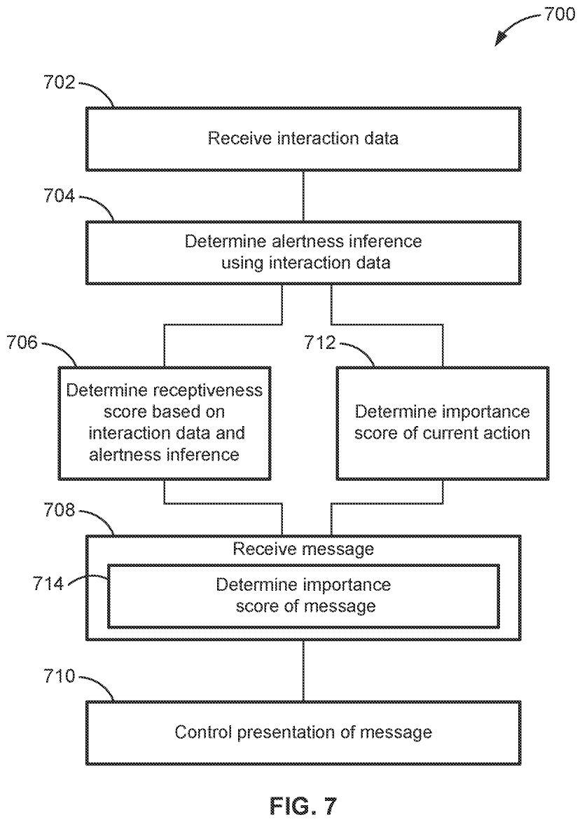

FIG. 7 is a flowchart depicting a process 700 for controlling presentation of a message based on a receptiveness score, according to certain aspects of the present disclosure. Process 700 can be performed by system 100 of FIG. 1, such as by a computing device (e.g., the one or more computing devices 120 of FIG. 1).

At block 702, interaction data can be received. At block 704, an alertness inference can be determined. Receiving interaction data at block 702 and determining an alertness inference at block 704 can be similar to receiving interaction data at block 506 and determining an alertness inference at block 514 of FIG. 5, respectively.

At block 706, a receptiveness score can be determined based on interaction data from block 702 and the alertness inference from block 704. The receptiveness score can be an indication as to how receptive the user is expected to be to a particular message. For example, a user concentrating hard (e.g., with a very high level of alertness) while interacting with an important app (e.g., an app dedicated to work email) may be very unreceptive to messages in general or certain particular messages (e.g., advertising content or text messages from a distant acquaintance). As well, a user with a very low level of alertness (e.g., while drowsy or possibly falling asleep) may not be very receptive to new messages. Alternatively, a with a moderate level of alertness (e.g., while casually interacting with the device), a user may be receptive to receiving the message.

Determining the receptiveness score can include applying one or more weighted formulae to the received interaction data and/or determined alertness inference to generate the receptiveness score. In some cases, generating the receptiveness score can include applying the inference data and/or alertness inference to an algorithm, such as a machine learning algorithm or deep neural network, to generate the receptiveness score. In some cases, determining the receptiveness score can include accessing supplemental information (e.g., supplemental information received at block 513 of FIG. 5).

The receptiveness score determined at block 706 can take any suitable form. In some cases, the receptiveness score can be generated, stored, and/or output as a number, such as a number in a range extending from non-receptive to fully receptive. Any suitable scale can be used, such as a numerical scale from 0 to 100, with higher numbers indicating a greater level of receptiveness. Other scoring techniques can be used. In some cases, a receptiveness score can include a receptiveness categorization, which can include lumping adjacent levels of receptiveness into the same overall receptiveness category. For example, a receptiveness score including a receptiveness categorization can categorize the levels of receptiveness into various enumerated levels (e.g., “non-receptive,” “mildly receptive,” “moderately receptive,” “strongly receptive,” and “fully receptive”).

In some cases, a receptiveness score as determined at block 706 can be a score associated with a user's overall receptiveness to messages in general. In some cases, however, a receptiveness score determined at block 706 can be a score associated with a particular message (e.g., a particular message or a source of a particular message). In such cases, determining the receptiveness score at block 706 may occur after receiving a message at block 708.

In some cases, an importance score of a current action can be determined at block 712. The current action can be an action associated with the user's interaction with the computing device. In some cases, the current action can be an app of other piece of software currently being used by the computing device. In some cases, the current action can be a message (e.g., a notification or text message) to which the user is responding. In some cases, the current action can be a type of task being performed by the user (e.g., word processing, texting, playing a game). Determining the importance score at block 712 can include associating the importance score with the action, such as associating the importance score with the respective app, software, message, type of task, or other element of the action. In some cases, determining the importance score at block 712 can include associating the importance score with any potential source of a future message, such as an app or an individual.

Determining an importance score at block 712 can include using the interaction data from block 702 and/or the alertness inference from block 704, respectively. For example, a user exhibiting high alertness while performing certain actions (e.g., using a particular app, performing a certain type of action, interacting with a message from a particular individual) may be indicative that the action in question has a high level of importance. However, a user exhibiting low alertness while performing other actions may be indicative that the other actions have a lower level of importance. Thus, an importance score of a current action can be determined based on the alertness inference and interaction data, as well as any previous importance scores for that current action or any other importance scores associated with the current action (e.g., an increase in the importance score associated with a word-processing type of task can be used to infer an increase in the importance associated with various specific word-processing apps).

Determining the importance score can include applying one or more weighted formulae to the received interaction data and/or determined alertness inference to generate the importance score. In some cases, generating the importance score can include applying the inference data and/or alertness inference to an algorithm, such as a machine learning algorithm or deep neural network, to generate the importance score. In some cases, determining the importance score can include accessing supplemental information (e.g., supplemental information received at block 513 of FIG. 5).

The importance score determined at block 712 can take any suitable form. In some cases, the importance score can be generated, stored, and/or output as a number, such as a number in a range extending from not-at-all-important to extremely important. Any suitable scale can be used, such as a numerical scale from 0 to 100, with higher numbers indicating a greater level of importance. Other scoring techniques can be used. In some cases, an importance score can include a importance categorization, which can include lumping adjacent levels of importance into the same overall importance category. For example, an importance score including a importance categorization can categorize the levels of importance into various enumerated levels (e.g., “not-at-all-important,” “mildly important,” “moderately important,” “strongly important,” and “extremely important”).

In some cases, determining the importance score at block 712 can occur as part of determining the receptiveness score at block 706. For example, determining the receptiveness score at block 706 can make use of an importance score determined at block 712, such as to determine whether or not the user is likely to be receptive to a message. For example, a user engaged in a highly important action may not be receptive to a new message, whereas user engaged in a not-important action may be more receptive to a new message. Additionally, in some cases, either of block 706 or block 712 can be excluded form process 700. For example, a receptiveness score can be determined at block 706 without any importance score being determined. For another example, an importance score can be determined at block 712 without any receptiveness score being determined.

At block 708, a message can be received. Receiving a message at block 708 can be similar to receiving a message at block 606 of FIG. 6. In some cases, receiving the message at block 708 can optionally include determining an importance score of the message at block 714. Determining the importance score of a message can include identifying a source of the message and applying an importance score associated with the source of the message to the message. For example, in previous instances of determining an importance score of an action (e.g., previous instances of block 712), the interaction data and/or alertness inferences may identify that a user generally responds very quickly to text messages from individual A, but generally dismisses or ignores text messages from individual B. In such cases, individual A may be associated with a high importance score and individual B may be associated with a low importance score. When determining the importance score of the message at block 714, an importance score associated with the source of the message can be used, such that a new message from individual A would be given a high importance score and a new message from individual B would be given a low importance score.

At block 710, presentation of the message received at block 708 can be controlled. Controlling presentation of the message can include presenting or not presenting the message, as well as presenting the message using any particular presentation scheme (e.g., presenting using a normal presentation scheme or an alternate or adjusted presentation scheme). Control of the presentation of the message at block 710 can be based on the receptiveness score from block 706 and/or the importance score of block 712. In some cases, control of the presentation of the message at block 710 can additionally be based on the importance score of the message as determined at block 714.

For example, if a low receptiveness score is determined at block 706, presentation of the message received at block 708 may be controlled at block 710 to be withheld, potentially to be re-delivered at a later time. In another example, a message received at block 708 may be used as input to determining a message-specific receptiveness score at block 706, such that once the message-specific receptiveness score at block 706 surpasses a threshold value, the message is presented (e.g., presentation is triggered) at block 710.

In some cases where an importance score of the message is determined at block 714, controlling presentation of the message at block 710 can involve presenting the message only when the importance score of the message is at or higher than an importance score associated with a current activity (e.g., as determined at block 712).

In some cases, determining an importance score of a message at block 714 can include determining whether the message is essential or non-essential. In some cases, controlling presentation of the message at block 710 can include always presenting essential messages or presenting essential messages using a particular presentation scheme (e.g., a presentation scheme including loud audio alerts, strong visual indications, and/or strong haptic feedback). Determining whether a message is essential or non-essential can be based on the importance score of the message, such that any message having an importance score over a particular threshold may be deemed essential. In some cases, determining whether a message is essential or non-essential can include analyzing the content of the message, such as to search for words or other content indicative that the message should be deemed essential.

While described with reference to process 700, determining whether or not a message is important can occur in any suitable process, and control of presentation of a message can be further informed by the determination of whether or not the message is essential. For example, in some cases, receiving the second message at block 516 of FIG. 5 can include determining whether or not the message essential (e.g., via determining an importance score or otherwise). In such an example, altering presentation of the second message at block 517 can be additionally based on the determination of whether or not the message is essential. For example, presentation of the second message may be altered only when the message is non-essential, or if the second message is essential, presentation may be altered to use a particular presentation scheme suitable for essential messages (e.g., with loud audio alerts, strong visual indications, and/or strong haptic feedback). Likewise, altering presentation at block 517 of FIG. 5 can be additionally based on an importance score associated with the message.

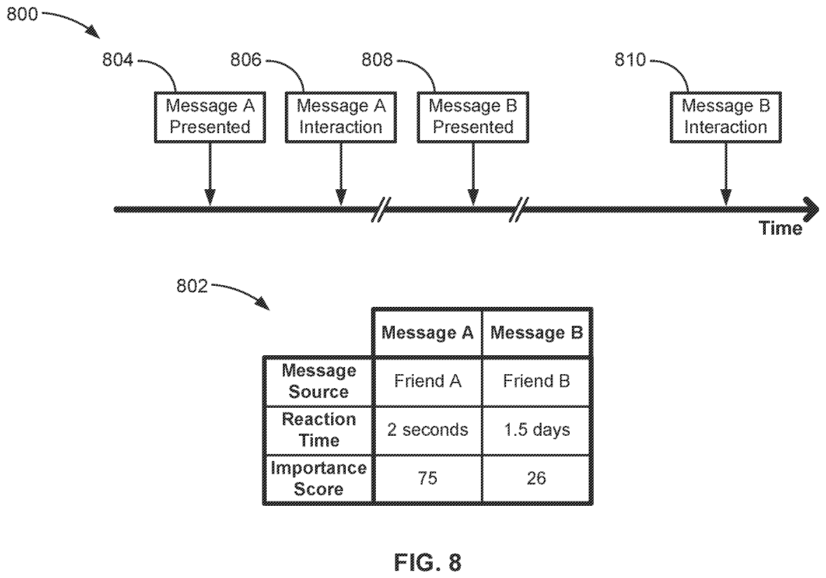

FIG. 8 is a combination timeline 800 and table 802 depicting reaction times for messages and resulting importance scores, according to certain aspects of the present disclosure. Timeline 800 includes indicators of messages being presented and reacted to by a user of a computing device, such as any of the one or more computing devices 120 of FIG. 1. For example, the messages can be incoming text messages from others, such as friends of the user using the computing device. FIG. 8 can depict a visual representation of determining an importance score associated with an action (e.g., responding to a message), as described with reference to block 712 of FIG. 7.

At time 804, message A can be presented on the computing device. A short time thereafter (e.g., within a second, a few seconds, a minute, or the like), at time 806, the user can interact with message A, such as by responding to the message.

At time 808, message B can be presented on the computing device. A relatively long time thereafter (e.g., within a few days, or the like), at time 810, the user can interact with the message B, such as by responding to the message. In some cases, the user may interact with message B at a time between time 808 and 810 to ignore or dismiss a notification of the message, in which case such ignoring or dismissal can either be ignored for purposes of determining an importance score or can be used to help infer an appropriate importance score (e.g., dismissal or ignoring of a notification may indicate the notification is not important).

The interactions depicted in timeline 800 are shown in table 802, along with examples of resultant importance scores. As shown in table 802, the source of message A can be friend A, and the reaction time (e.g., time elapsed between time 804 and time 806) can be 2 seconds. Because of this relatively speedy reaction time, the system can infer that messages from friend A are important and associate the message and/or message source with a relatively high importance score, such as 75 out of 100. Message B, however, with a source of friend B, is shown with a reaction time of 1.5 days. Because of this relatively long reaction time, the system can infer that messages from friend B are not important and associate the message and/or message source with a relatively low importance score, such as 26 out of 100.

As described with reference to FIG. 7, these importance scores can be later used at block 714 to determine an importance score of a new message. For example, if a new message were to come in, the system would automatically apply a high importance score (e.g., 75 out of 100) if the message came from friend A, but automatically apply a low importance score (e.g., 26 out of 100) if the message came from friend B.

While described with reference to friends in FIG. 8, and other message source can be used. Additionally, while described in FIG. 8 with reference to presenting text messages and interacting with text messages, any other presentation and/or interaction with messages can be used. For example, quickly updating an app after being presented with a notice about an available update to the app can be used to infer that the app may have a high importance score.

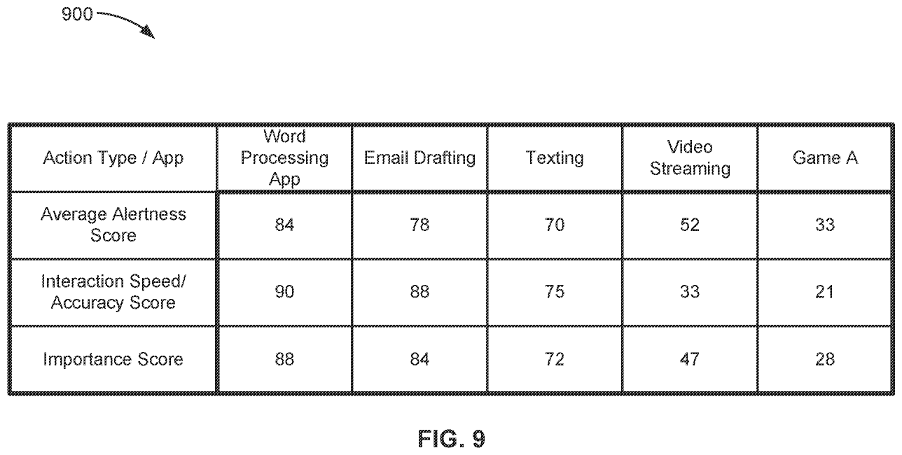

FIG. 9 is a table 900 depicting alertness scores, interaction speed/accuracy scores, and importance scores for various actions on a computing device, according to certain aspects of the present disclosure. The table 900 of FIG. 9 can be a visual representation of determining an importance score associated with an action, as described with reference to block 712 of FIG. 7.

Table 900 can include indicators for various actions, such as an app (e.g., Game A) or a type of action (e.g., Email Drafting). As part of determining an importance score, the system can make use of an alertness inference, which is represented by the “Average Alertness Score” line on table 900. As part of determining an importance score, the system can make use of interaction data, which is represented by the “Interaction Speed/Accuracy Score” line on table 900. Based on the alertness inference(s) and/or interaction data, the system can determine an importance score for various actions, examples of which are shown on the “Importance Score” line on table 900.

In the example shown in FIG. 9, this particular user is very alert while using a word processing app, reacting to prompts quickly and/or maintaining high button-press accuracy during use, as evident by the relatively high average alertness score of 84 out of 100 and the relatively high interaction speed/accuracy score of 90 out of 100. Therefore, the system can determine that the importance score associated with the word processing app should be relatively high, such as 88 out of 100. Additionally, this particular user is not very alert while playing Game A, reacting to prompts slowly and/or exhibiting low button-press accuracy during use, as evident by the relatively low average alertness score of 33 out of 100 and the relatively low interaction speed/accuracy score of 21 out of 100. Therefore, the system can determine that the importance score associated with Game A should be relatively low, such as 28 out of 100.

In such an example, while the user is interacting with the word processing app, and especially if the user is exhibiting a high level of alertness based on a current alertness inference, messages originating from Game A may be withheld or presented in a different fashion, since the importance score associated with Game A is much lower than the importance score associated with the word processing app. Likewise, while the user is interacting with Game A, optionally regardless of the user's current alertness inference, messages originating from the word processing app may be presented, since the importance score associated with the word processing app is much higher than the importance score associated with Game A.

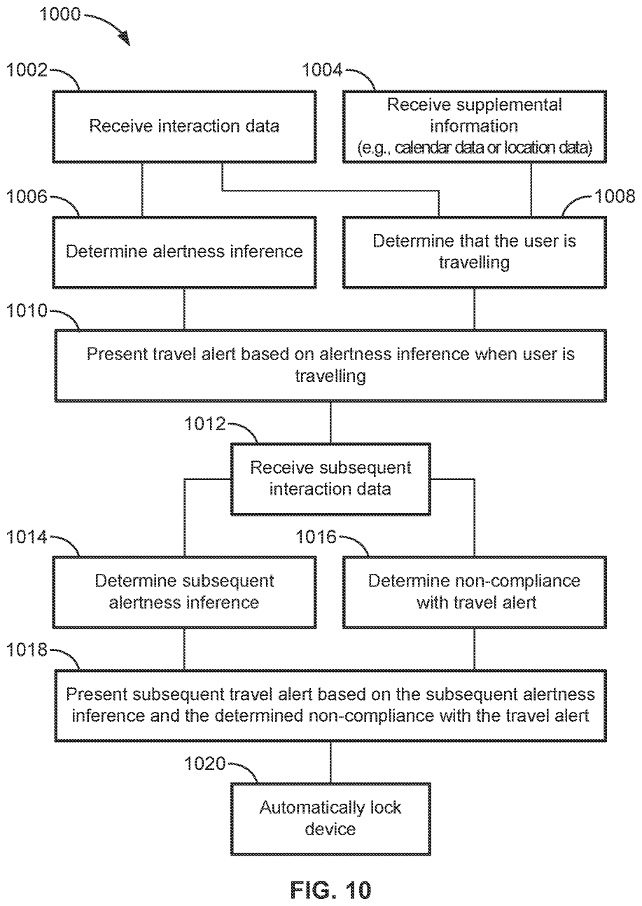

FIG. 10 is a flowchart depicting a process 1000 for controlling presentation of a travel alert based on an alertness inference, according to certain aspects of the present disclosure. Process 1000 can be performed by system 100 of FIG. 1, such as by a computing device (e.g., the one or more computing devices 120 of FIG. 1).

At block 1002, interaction data can be received. At block 1006, an alertness inference can be determined. Receiving interaction data at block 1002 and determining an alertness inference at block 1006 can be similar to receiving interaction data at block 506 and determining an alertness inference at block 514 of FIG. 5, respectively.

At optional block 1004, supplemental information can be received. Supplemental information can include information associated with travel of a user. Examples of suitable supplemental information include calendar data, location data, ticket data, itinerary data, and the like.

At block 1008, a determination can be made that the user is travelling. This determination can be made using the interaction data from block 1002 and/or from optional supplemental information received at block 1004. For example, presence of an electronic ticket can be indicative that the user is or will be travelling as indicated on the ticket. In some cases, certain interaction data can be analyzed to infer that the user is travelling, such as motion data indicative of the user being a passenger in a train or airplane. In some cases, both interaction data and supplemental information can be used to determine that the use is travelling.

At block 1010, a travel alert can be presented based on the alertness inference from block 1006 and the determination that the user is travelling from block 1008. The travel alert can be any suitable alert. In an example, when the alertness inference determined at block 1006 is indicative that the user is drowsy, the alert can warn the user to stow and/or secure the computing device, so as to avoid the user dropping or otherwise losing the computing device if the user falls asleep. Other alerts can be used, including based on other types of alertness inferences.

In some cases, process 1000 can include only blocks 1002, 1004, 1006, 1008, and 1010, although that need not always be the case.

At block 1012, subsequent interaction data can be received. At block 1014, a subsequent alertness inference can be determined. Receiving subsequent interaction data at block 1012 and determining a subsequent alertness inference at block 1014 can be similar to receiving subsequent interaction data at block 610 and determining a subsequent alertness inference at block 612 of FIG. 6.

Optionally, at block 1016, non-compliance with the travel alert presented at block 1010 can be determined. For example, if a travel alert was presented to stow or otherwise secure the computing device, non-compliance with that alert can be determined by analyzing the subsequent interaction data to identify that the computing device has not been stowed and/or secured. For example, subsequent interaction data may be indicative that the device is still being held by the user and/or is slipping from the user's hand.

At block 1018, a subsequent travel alert can be presented based on the subsequent alertness inference and the determined non-compliance with the travel alert. For example, if the subsequent alertness interface determined at block 1014 is indicative that the user has fallen asleep, and/or non-compliance with the travel alert is determined at block 1016, the subsequent travel alert presented at block 1018 can be in the form of an alarm to awaken the user to facilitate compliance with the travel alert of block 1010. In some cases, at optional block 1020, alternative to or in addition to presenting the subsequent travel alert at block 1018, the computing device can be locked. In some cases, optional block 1020 could include implementing a change to app settings and/or device settings, and/or initiating a communication with a cloud location or finding service.

For example, a user watching a movie on a smartphone may start to doze off during the movie. The computing device may provide the first travel alert at block 1010 to warn the user to secure the smartphone before the user falls asleep, but the user may fail to do so. Eventually, after determining that the user has fallen asleep at block 1014, a second travel alert can be presented to attempt to awaken the user and have the user secure the smartphone. Finally, in addition to or alternative to the second travel alert, the system can automatically lock the smartphone, such as to prevent illicit access by third parties while the user is asleep. Without certain aspects of the present disclosure, if a user falls asleep while watching content on a smartphone while travelling, the smartphone will likely keep playing until the end of the content (e.g., end of a movie, end of a playlist of content, or end of a season of episodes), remaining in an unlocked state and available for illicit access by third parties.

In some cases, other travel-related actions can be taken.

In an example, the travel alert presented at block 1010 can be an alarm to indicate the user is approaching a destination. In such a case, the alarm can be automatically set based on an inference that the user has fallen asleep or otherwise has insufficient levels of alertness to exit the vehicle at the destination. Such an alarm can be set using the supplemental information from block 1004, such as based on analyzing the supplemental information do identify a likely destination and/or a likely time when the user will reach the destination.

In some cases, supplemental information can further be used to present an alert or otherwise take an action when a determined alertness inference (e.g., low alertness, such as falling asleep) is detected while threshold relative movement is detected between a first device and a second device. In an example, if a first device is moved apart from a second device when a user is detected at a low level or alertness, a tracking process can be initiated, such as initiation of a finding service connection. For example, a user may fall asleep when using a mobile device such as a smartphone, while wearing a secondary device such as a smart watch. If the smartphone is taken and moved away from the user (e.g., the user's smart watch), an alert may be triggered. Such an alert can be triggered even if the phone and watch are still within wireless range of each other (e.g., using the alertness inference and/or other sensors, such as the IMU to detect unexpected motion). In some cases, the second device might be a smart tag or tracker, such as a smart tracker attached to, placed in, or otherwise incorporated with a bag or wallet. Unexpected motion or change of position or location of the first or second device, or a relative change in position, may trigger an audible or inaudible alert when combined with a determination of a low level of alertness.

In another example, where two devices and an alertness level are available, if the user is drowsy and moves to a second place (such as getting up and walking to door of a bus), even though the devices are still in wireless range, for the lower alertness level, the system may proactively trigger a reminder or alert to one or both devices and to the user, such as to remind them to, for example, very rapidly find an accidentally forgotten device while there is still time to do so.

In some cases, any of processes 500, 600, 700, and 1000 of FIGS. 5-7 and 10, respectively, as well as any elements (e.g., blocks) of the processes, can be combined with one another as appropriate. For example, while process 500 does not outline determining a receptiveness score or importance score, such a determination can be included in a variation of process 500. Other combinations can be used.

In some implementations, processes 500, 600, 700, and 1000 of FIGS. 5-7 and 10, respectively, can be performed by a supervised or unsupervised algorithm. For instance, the system may utilize more basic machine learning tools including (1) decision trees (“DT”), (2) Bayesian networks (“BN”), (3) artificial neural network (“ANN”), or (4) support vector machines (“SVM”). In other examples, deep learning algorithms or other more sophisticated machine learning algorithms, e.g., convolutional neural networks (“CNN”), recurrent neural networks (“RNN”), or capsule networks (“CapsNet”) may be used.

DT are classification graphs that match user input data to device data at each consecutive step in a decision tree. The DT program moves down the “branches” of the tree based on the user input to the recommended device settings (e.g., First branch: Did the device data include certain sleep states? yes or no. Branch two: Did the device data include certain time stamps? yes or no, etc.).

Bayesian networks (“BN”) are based on likelihood something is true based on given independent variables and are modeled based on probabilistic relationships. BN are based purely on probabilistic relationships that determine the likelihood of one variable based on another or others. For example, BN can model the relationships between device data, user input data, and any other information as contemplated by the present disclosure.

Artificial neural networks (“ANN”) are computational models inspired by an animal's central nervous system. They map inputs to outputs through a network of nodes. However, unlike BN, in ANN the nodes do not necessarily represent any actual variable. Accordingly, ANN may have a hidden layer of nodes that are not represented by a known variable to an observer. ANNs are capable of pattern recognition. Their computing methods make it easier to understand a complex and unclear process that might go on during determining a symptom severity indicator based a variety of input data.

Support vector machines (“SVM”) came about from a framework utilizing of machine learning statistics and vector spaces (linear algebra concept that signifies the number of dimensions in linear space) equipped with some kind of limit-related structure. In some cases, they may determine a new coordinate system that easily separates inputs into two classifications. For example, a SVM could identify a line that separates two sets of points originating from different classifications of events.

Deep neural networks (DNN) have developed recently and are capable of modeling very complex relationships that have a lot of variation. Various architectures of DNN have been proposed to tackle the problems associated with algorithms such as ANN by many researchers during the last few decades. These types of DNN are CNN (Convolutional Neural Network), RBM (Restricted Boltzmann Machine), LSTM (Long Short Term Memory) etc. They are all based on the theory of ANN. They demonstrate a better performance by overcoming the back-propagation error diminishing problem associated with ANN.

Machine learning models require training data to identify the features of interest that they are designed to detect. For instance, various methods may be utilized to form the machine learning models, including applying randomly assigned initial weights for the network and applying gradient descent using back propagation for deep learning algorithms. In other examples, a neural network with one or two hidden layers can be used without training using this technique.

In some implementations, the machine learning model can be trained using individual data and/or data that represents a certain user. In other examples, the data will only be updated with individual data and historical data from a plurality of users may be input to train the machine learning algorithm.

While the present disclosure has been described with reference to one or more particular embodiments or implementations, those skilled in the art will recognize that many changes may be made thereto without departing from the spirit and scope of the present disclosure. Each of these implementations and obvious variations thereof is contemplated as falling within the spirit and scope of the present disclosure. It is also contemplated that additional implementations according to aspects of the present disclosure may combine any number of features from any of the implementations described herein.

1. A method comprising:
receiving a first message intended for presentation by a computing device;
presenting the first message on the computing device using a first presentation scheme in response to receiving the first message;
receiving interaction data associated with a user interacting with the computing device;
determining an alertness inference based on the interaction data, wherein the alertness inference is indicative of a degree of alertness of the user;
receiving a second message intended for presentation by the computing device; and
altering presentation of the second message on the computing device based on the determined alertness inference, wherein altering the presentation of the second message comprises presenting the second message using a second presentation scheme.

2. The method of claim 1, wherein presenting the first message using the first presentation scheme comprises presenting the first message with an audible alert, and wherein presenting the second message using the second presentation scheme comprises presenting the second message without an audible alert.

3. The method of claim 1, wherein the interaction data is collected by the computing device.

4. The method of claim 1, wherein the interaction data comprises one or more of biometric data of the individual, inertia data of the computing device, and software-usage data of the computing device.

5. The method of claim 1, wherein the interaction data comprises biometric data of the individual, and wherein the biometric data comprises one or more of eye focus data, blink rate data, and head sway data.

6. The method of claim 1, wherein the interaction data comprises biometric data of the individual, wherein the biometric data comprises biomotion data, and wherein the biomotion data comprises torso movement, limb movement, respiration, head movement, eye movement, hand movement, finger movement, or cardiac movement.

7. The method of claim 1, wherein the biometric data is collected using (a) a user-facing camera of the computing device or (b) a radiofrequency sensor.

8. (canceled)

9. The method of claim 1, wherein the interaction data further comprises inertia data of the computing device or software-usage data of the computing device, and wherein determining an alertness inference based on the interaction data comprises determining an alertness inference based on one of the biometric data, the inertia data, and the software-usage data; and confirming the alertness inference using another of the biometric data, the inertia data, and the software-usage data.

10. The method of claim 1, wherein the interaction data comprises software-usage data, and wherein determining the alertness inference comprises generating an alertness score of the user based on at least one of a speed of interaction of the user and an accuracy of interaction of the user.

11. The method of claim 1, wherein presenting the first message comprises applying a notification rule of the computing device to the first message when received, and wherein altering presentation of the second message comprises modifying the notification rule of the computing device.

12. The method of claim 11, wherein modifying the notification rule occurs prior to receiving the second message.

13. The method of claim 1, further comprising analyzing the second message to determine that the second message is non-essential, wherein altering presentation of the second message is based on the determined alertness inference and the determination that the second message is non-essential.

14. The method of claim 1, further comprising receiving supplemental information associated with the user interacting with the computing device, wherein the supplemental information comprises at least one of a time of day, a geolocation, a time zone, power data from the computing device, or an ambient light level;
wherein determining the alertness inference is further based on the supplemental information.

15. The method of claim 1, wherein receiving interaction data comprises receiving first message interaction data associated with the user interaction with the presentation of the first message, the method further comprising determining an importance score associated with the first message based on the first message interaction data, wherein receiving the second message comprises assigning a presumed importance score to the second message based on the importance score associated with the first message, and wherein altering presentation of the second message is further based on the presumed importance score of the second message.

16. The method of claim 1 further comprising:
receiving subsequent interaction data associated with the user subsequently interacting with the computing device;
determining a subsequent alertness inference based on the subsequent interaction data, wherein the subsequent alertness inference is indicative of a subsequent degree of alertness of the user that is different than the degree of alertness of the user;
presenting the second message according to the first presentation scheme or a third presentation scheme in response to the subsequent alertness inference.

17. The method of claim 16, wherein the second message comprises advertising content, the method further comprising:
determining a receptiveness score based on the alertness inference and the interaction data, wherein the receptiveness score is indicative of receptiveness to advertising content, wherein alerting presentation of the second message comprises withholding presentation of the second message when the receptiveness score is below a threshold score;
determining a subsequent receptiveness score based on the subsequent alertness inference and the subsequent interaction data, wherein presenting the second message according to the first presentation scheme in response to the subsequent alertness inference occurs when the subsequent receptiveness score is at or above the threshold score.

18. The method of claim 17, wherein determining a receptiveness score comprises determining an importance score associated with an action being taken by the user on the computing device based on received interaction data, wherein the importance score is indicative of a perceived importance of the action to the user based on the received interaction data.

19. The method of claim 18, wherein the action is associated with a particular app on the computing device, and wherein the importance score associated with the action is an importance score associated with the app.

20. The method of claim 1, wherein the second message comprises advertising content, the method further comprising selecting a route of presentation based on the alertness inference and the received interaction data, wherein altering presentation of the second message comprises presenting the second message using the second presentation scheme, and wherein the second presentation scheme uses the selected route of presentation.

21. (canceled)

22. The method of claim 1, further comprising:
determining that the user is travelling based on the received interaction data, calendar data, or location data; and
presenting a travel alert based on the alertness inference.

23-25. (canceled)

26. The method of claim 1, further comprising:
determining that the user is travelling based on the received interaction data, calendar data, or location data, wherein determining that the user is travelling comprises identifying a presumed destination;
determining that the user is asleep based on the alertness inference; and
automatically setting an alarm after determining that the user is asleep, wherein the alarm is set to wake the user prior to arrival at the presumed destination.

27. The method of claim 1, further comprising:
determining an importance score associated with an action being taken by the user on the computing device at the time the second message is received based on the received interaction data and the determined alertness inference; and
determining an importance score associated with the second message, wherein altering presentation of the second message is further based on comparing the importance score of the second message with the importance score of the action being taken by the user.

28-29. (canceled)

30. The method of claim 1, wherein the computing device is a mobile device comprising an inertia measurement unit for obtaining inertia data and a user-facing camera for obtaining biometric data.

31. (canceled)

32. The method of claim 1, further comprising supplying air to the user by a respiratory therapy device, the respiratory therapy device being communicatively coupled to the computing device, wherein the interaction data is associated with the user interacting with i) a respiratory therapy device companion app on the computing device; ii) an interactive display of the respiratory therapy device; or iii) a combination of i and ii.

33-34. (canceled)

35. A method, comprising:
receiving a message intended for presentation to a user by a computing device; and
altering presentation of the message on the computing device based on a determined alertness inference, wherein the alertness inference is indicative of a degree of alertness of the user, wherein altering the presentation of the message comprises presenting the message using an altered presentation scheme, and wherein the alertness inference is determined based on:
receiving a prior message intended for presentation by the computing device;
presenting the prior message on the computing device using a presentation scheme in response to receiving the prior message, wherein the altered presentation scheme is different than the presentation scheme;
receiving interaction data associated with the user interacting with the computing device; and
determining the alertness inference based on the interaction data.

36-39. (canceled)
